(19) United States
(12) Patent Application Publication
(10) Pub. No.: US 2005/0106576 A1
(43) Pub. Date: May 19, 2005

(54) Methods of using cleavable solid phases for isolating nucleic acids

(76) Inventors: Hashem Akhavan-Tafti, Howell, MI (US); Renuka de Silva, Northville, MI (US); Nicole M. Cilli, Pontiac, MI (US); William G. Cripps, Ann Arbor, MI (US); Richard S. Handley, Canton, MI (US); Elizabeth A. O'Connor, Dearborn Hts., MI (US); Lekkala V. Reddy, Ann Arbor, MI (US); Sarada Siripurapu, Novi, MI (US)

Correspondence Address: LUMIGEN, INC., 22900 W. EIGHT MILE ROAD, SOUTHFIELD, MI, 48034, US

(21) Appl. No.: 10/714,763

(22) Filed: Nov. 17, 2003

(51) Int. Cl. C12Q 1/68; C07H 21/04

(52) U.S. Cl. 435/6; 536/25.4

(57) ABSTRACT

Solid phase materials for binding nucleic acids and methods of their use are disclosed. The materials feature a cleavable linker portion which can be cleaved to release bound nucleic acids. The solid phase materials comprise a solid support portion comprising a matrix selected from silica, glass, insoluble synthetic polymers, and insoluble polysaccharides to which is attached a nucleic acid binding portion for attracting and binding nucleic acids, the nucleic acid binding portion (NAB) being linked by a cleavable linker portion to the solid support portion. Preferred nucleic acid binding portions comprise a ternary or quaternary onium group. The materials can be in the form of microparticles, fibers, beads, membranes, test tubes or microwells and can further comprise a magnetic core portion. Methods of binding nucleic acids using the cleavable solid supports are disclosed as are their use in methods of isolating or purifying nucleic acids.

### FIELD OF THE INVENTION

The present invention relates to the use of novel solid phase materials in methods of binding, isolating, and purifying nucleic acids.

### BACKGROUND OF THE INVENTION

Molecular diagnostics and modern techniques in molecular biology (including reverse transcription, cloning, restriction analysis, amplification, and sequence analysis), require that nucleic acids used in these techniques be substantially free of contaminants and interfering substances. Undesirable contaminants include macromolecular substances such as enzymes, other types of proteins, polysaccharides, polynucleotides, oligonucleotides, nucleotides, lipids, low molecular weight enzyme inhibitors, or non-target nucleic acids, enzyme cofactors, salts, chaotropes, dyes, metal salts, buffer salts and organic solvents.

Obtaining target nucleic acid substantially free of contaminants for molecular biological applications is difficult due to the complex sample matrix in which target nucleic acids are found. Such samples include, e.g., cells from tissues, cells from bodily fluids, blood, bacterial cells in culture, agarose gels, polyacrylamide gels, or solutions resulting from amplification of target nucleic acids. Sample matrices often contain significant amounts of contaminants which must be removed from the nucleic acid(s) of interest before the nucleic acids can be used in molecular biological or diagnostic techniques.

Conventional techniques for isolating target nucleic acids from mixtures produced from cells and tissues as described above, require the use of hazardous chemicals such as phenol, chloroform, and ethidium bromide. Phenol/chloroform extraction is used in such procedures to extract contaminants from mixtures of target nucleic acids and various contaminants. Alternatively, cesium chloride-ethidium bromide gradients are used according to methods well known in the art. See, e.g., Molecular Cloning, ed. by Sambrook et al. (1989), Cold Spring Harbor Press, pp. 1.42-1.50. The latter methods are generally followed by precipitation of the nucleic acid material remaining in the extracted aqueous phase by adding ethanol or 2-propanol to the aqueous phase to precipitate nucleic acid. The precipitate is typically removed from the solution by centrifugation, and the resulting pellet of precipitate is allowed to dry before being resuspended in water or a buffer solution for further use.

Simpler and faster methods have been developed which use various types of solid phases to separate nucleic acids from cell lysates or other mixtures of nucleic acids and contaminants. Such solid phases include chromatographic resins, polymers and silica or glass-based materials in various shapes and forms such as fibers, filters and coated containers. When in the form of small particulates, magnetic cores are sometimes provided to assist in effecting separation.

One type of solid phase used in isolating nucleic acids comprises porous silica gel particles designed for use in high performance liquid chromatography (HPLC). The surface of the porous silica gel particles is functionalized with anion-exchangers to exchange with plasmid DNA under certain salt and pH conditions. See, e.g. U.S. Pat. Nos. 4,699,717, and 5,057,426. Plasmid DNA bound to these solid phase materials is eluted in an aqueous solution containing a high concentration of a salt. The nucleic acid solution eluted therefrom must be treated further to remove the salt before it can be used in downstream processes.

Other silica-based solid phase materials comprise controlled pore glass (CPG), filters embedded with silica particles, silica gel particles, diatomaceous earth, glass fibers or mixtures of the above. Each silica-based solid phase material reversibly binds nucleic acids in a sample containing nucleic acids in the presence of chaotropic agents such as sodium iodide (NaI), guanidinium thiocyanate or guanidinium chloride. Such solid phases bind and retain the nucleic acid material while the solid phase is subjected to centrifugation or vacuum filtration to separate the matrix and nucleic acid material bound thereto from the remaining sample components. The nucleic acid material is then freed from the solid phase by eluting with water or a low salt elution buffer. Commercially available silica-based solid phase materials for nucleic acid isolation include, e.g., Wizard™ DNA purification systems products (Promega, Madison, Wis.), the QiaPrep™ DNA isolation systems (Qiagen, Santa Clarita, Calif.), High Pure (Roche), and GFX Micro Plasmid Kit, (Amersham).

Polymeric resins in the form of particles are also in widespread use for isolation and purification of nucleic acids. Carboxylate-modified polymeric particles (Bangs, Agencourt) polymers having quaternary ammonium head groups are disclosed in European Patent Application Publ. No. EP 1243649A1. The polymers are inert carrier particles having covalently attached linear non-crosslinked polymers. This type of polymeric solid phase is commonly referred to as a tentacle resin. The linear polymers incorporate quaternary tetraalkylammonium groups. The alkyl groups are specified as methyl or ethyl groups (Column 4, lines 52-55). Longer alkyl groups are deemed undesirable.

Other solid phase materials for binding nucleic acids based on the anion exchange principle are in present use. These include a silica based material having DEAE head groups (Qiagen) and a silica-NucleoBond AX (BD, Roche-Genopure) based on the chromatographic support described in EP0496822B1. Polymer resins with polymeric-trialkylammonium groups are disclosed in EP 1243649 (GeneScan). Carboxyl-modified polymers for DNA isolation are available from numerous suppliers. Nucleic acids are attracted under high salt conditions and released under low ionic strength conditions.

Magnetically responsive particles have also been developed for use as solid phases in isolating nucleic acids. Several different types of magnetically responsive particles designed for isolation of nucleic acids are known in the art and commercially available from several sources. Magnetic particles which reversibly bind nucleic acid materials directly include MagneSil™ particles (Promega). Magnetic particles are also known that reversibly bind mRNA via covalently attached avidin or streptavidin having an attached oligo dT tail for hybridization with the poly A tail of mRNA.

Various types of magnetically responsive silica-based particles are known for use as solid phases in nucleic acid binding isolation methods. One such particle type is a magnetically responsive glass bead, preferably of a controlled pore size available as Magnetic Porous Glass (MPG) particles from CPG, Inc. (Lincoln Park, N.J.); or porous magnetic glass particles described in U.S. Pat. Nos. 4,395,271; 4,233,169; or 4,297,337. Another type of magnetic particle useful for binding and isolation of nucleic acids is produced by incorporating magnetic materials into the matrix of polymeric silicon dioxide compounds. (German Patent DE4307262A1).

Particles or beads having inducible magnetic properties comprise small particles of transition metals such as iron, nickel, copper, cobalt and manganese to form metal oxides which can be caused to have transitory magnetic properties in the presence of magnet. These particles are termed paramagnetic or superparamagnetic. To form paramagnetic or superparamagnetic beads, metal oxides have been coated with polymers which are relatively stable in water. U.S. Pat. No. 4,554,088 discloses paramagnetic particles comprising a metal oxide core surrounded by a coat of polymeric silane. U.S. Pat. No. 5,356,713 discloses a magnetizable microsphere comprised of a core of magnetizable particles surrounded by a shell of a hydrophobic vinylaromatic monomer. U.S. Pat. No. 5,395,688 discloses a polymer core which has been coated with a mixed paramagnetic metal oxide-polymer layer. Another method utilizes a polymer core to adsorb metal oxide such as for example in U.S. Pat. No. 4,774,265. Magnetic particles comprising a polymeric core particle coated with a paramagnetic metal oxide particle layer is disclosed in U.S. Pat. No. 5,091,206. The particle is then further coated with additional polymeric layers to shield the metal oxide layer and to provide a reactive coating. U.S. Pat. No. 5,866,099 discloses the preparation of magnetic particles by coprecipitation of mixtures of two metal salts in the presence of a protein to coordinate the metal salt and entrap the mixed metal oxide particle. Numerous exemplary pairs of metal salts are described. U.S. Pat. No. 5,411,730 describes a similar process where the precipitated mixed metal oxide particle is entrapped in dextran, an oligosaccharide.

Alumina (aluminum oxide) particles for irreversible capture of DNA and RNA is disclosed in U.S. Pat. No. 6,291,166. Bound nucleic acid is available for use in solid phase amplification methods such as PCR.

### SUMMARY OF THE INVENTION

It is another object of the present invention to provide solid phase materials comprising a cleavable linker for binding nucleic acids. It is a further object to provide such cleavable solid phase materials comprising a covalently linked nucleic acid binding group. It is another object of the present invention to provide methods for binding and releasing nucleic acids from the solid phase materials. It is another object of the present invention to provide methods of isolating and purifying nucleic acids using the solid phase materials of the present invention. A further object of the present invention is to provide solid phase materials which bind nucleic acids and resist release of the nucleic acids under most commonly used elution conditions. It is a further object to provide such solid phase materials which contain covalently linked ternary or quaternary onium groups. It is another object of the present invention to provide solid phase materials for binding nucleic acids and releasing the nucleic acids with compositions of the present invention. It is another object of the present invention to provide such reagent compositions for releasing bound nucleic acids from solid phase materials.

### BRIEF DESCRIPTION OF THE DRAWINGS

FIG. 1A depicts a schematic representation of a cleavable nucleic acid binding particle. FIG. 1B depicts a cleavable solid support binding a nucleic acid molecule.

FIG. 2 shows the binding and release of a nucleic acid using a cleavable nucleic acid binding particle.

FIG. 3 is an image of a gel of PCR amplified pUC18 plasmid DNA samples which had been adsorbed onto 10 mg of cleavable polymer beads, and eluted from washed beads before amplification.

FIG. 4 is an image of a gel of pUC18 DNA obtained by isolation from a cell lysate using cleavable beads of examples 13 and 19.

FIG. 5 is an image of a gel of DNA isolated from human blood samples using a cleavable solid support of the invention.

FIG. 6 is an image of a dot blot of DNA bound to a cleavable solid support of the invention having tributyl-phosphonium groups and released by Wittig reaction.

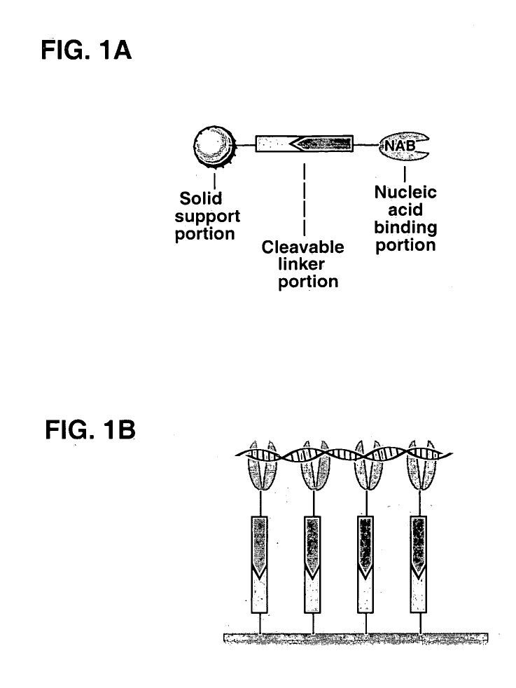

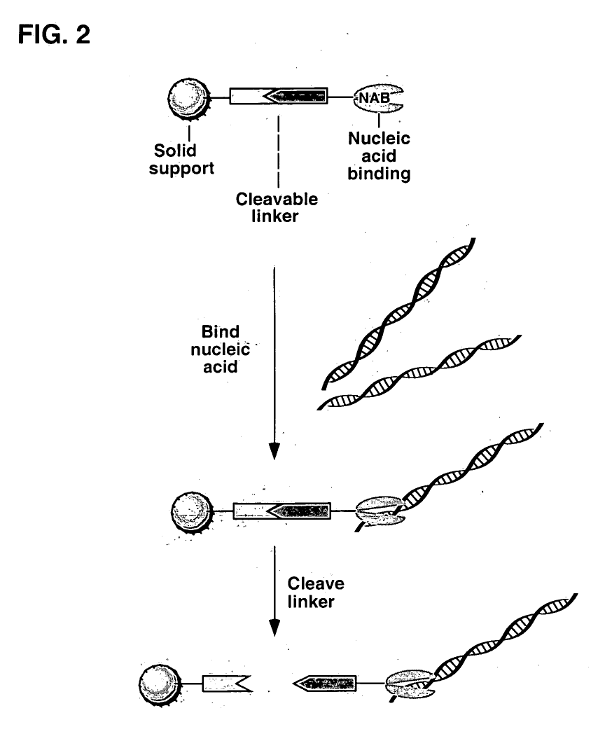

### DETAILED DESCRIPTION OF THE INVENTION

Applicants have developed new solid phase materials useful for capturing and binding nucleic acids from solutions and samples containing nucleic acids. The solid phase materials can be in the form of particles, microparticles, fibers, beads, membranes, and other supports such as test tubes and microwells. A defining characteristic of the new materials is the presence of a cleavable linker portion. The materials further comprise an nucleic acid binding group which permits capture and tight binding of nucleic acid molecules of varying lengths. Reaction of the solid phase materials with an agent that breaks the cleavable linker allows the release of bound nucleic acid from the solid phase. Novel methods of controllably releasing bound nucleic acid molecules form a further portion of the invention as do reagent compositions for releasing or eluting bound nucleic acid molecules from the solid phase materials.

Definitions

Alkyl—A branched, straight chain or cyclic hydrocarbon group containing from 1-20 carbons which can be substituted with 1 or more substituents other than H. Lower alkyl as used herein refers to those alkyl groups containing up to 8 carbons.

Aralkyl—An alkyl group substituted with an aryl group.

Aryl—An aromatic ring-containing group containing 1 to 5 carbocyclic aromatic rings, which can be substituted with 1 or more substituents other than H.

Magnetic particle—a particle, microparticle or bead that is responsive to an external magnetic field. The particle may itself be magnetic, paramagnetic or superparamagnetic. It may be attracted to an external magnet or applied magnetic field as when using ferromagnetic materials. Particles can have a solid core portion that is magnetically responsive and is surrounded by one or more non-magnetically responsive layers. Alternately the magnetically responsive portion can be a layer around or can be particles disposed within a non-magnetically responsive core.

Oligomer, oligonucleotide—as used herein will refer to a compound containing a phosphodiester internucleotide linkage and a 5′-terminal monophosphate group. The nucleotides can be the normally occurring ribonucleotides A, C, G, and U or deoxyribonucleotides, dA, dC, dG and dT.

Polynucleotide—A polynucleotide can be DNA, RNA or a synthetic DNA analog such as a PNA. Double-stranded hybrids of any of these three types of chains are also within the scope of the term.

Primer—refers to an oligonucleotide used to direct the site of ligation and is required to initiate the ligation process. Primers are of a length sufficient to hybridize stably to the template and represent a unique sequence in the template. Primers will usually be about 15-30 bases in length. Labeled primers containing detectable labels or labels which allow solid phase capture are within the scope of the term as used herein.

Template, test polynucleotide, and target are used interchangeably and refer to the nucleic acid whose length is to be replicated.

Sample—A fluid containing or suspected of containing nucleic acids. Typical samples which can be used in the methods of the invention include bodily fluids such as blood, plasma, serum, urine, semen, saliva, cell lysates, tissue extracts and the like. Other types of samples include solvents, seawater, industrial water samples, food samples and environmental samples such as soil or water, plant materials, cells originated from prokaryotes, eukaryotes, bacteria, plasmids and viruses.

Solid phase material—a material having a surface to which can attract nucleic acid molecules. Materials can be in the form of microparticles, fibers, beads, membranes, and other supports such as test tubes and microwells.

Substituted—Refers to the replacement of at least one hydrogen atom on a group by a non-hydrogen group. It should be noted that in references to substituted groups it is intended that multiple points of substitution can be present unless clearly indicated otherwise.

Applicants have developed solid phase materials which bind nucleic acids and have a cleavable linker portion which can be cleaved to release the bound nucleic acids. The materials can be in the form of microparticles, fibers, beads, membranes, and other supports such as test tubes and microwells that have sufficient surface area to permit efficient binding. Solid phase materials of the present invention in the form of microparticles can further comprise a magnetic core portion. Generally, particles and magnetically responsive microparticles are preferred in the present invention.

The solid phase nucleic acid binding materials of the present invention comprise a matrix which defines its size, shape, porosity, and mechanical properties, and covalently linked nucleic acid binding groups. The three most common kinds of matrix are silica or glass, insoluble synthetic polymers, and insoluble polysaccharides. The solid phase can further comprise a magnetically responsive portion.

Polymers are homopolymers or copolymers of one or more ethylenically unsaturated monomer units and can be crosslinked or non-crosslinked. Preferred polymers are polyolefins including polystyrene and the polyacrylic-type polymers. The latter comprise polymers of various substituted acrylic acids, amides and esters, wherein the acrylic monomer may or may not have alkyl substituents on the 2- or 3-carbon.

The nucleic acid binding groups contained in the solid phase binding materials of the present invention attract and bind nucleic acids, polynucleotides and oligo-nucleotides of various lengths and base compositions or sequences. Nucleic acid binding groups include carboxyl, amine and ternary or quaternary onium groups. Amine groups can be NH₂, alkylamine, and dialkylamine groups. Ternary or quaternary onium groups include quaternary trialkylammonium groups (—QR₃⁺), phosphonium groups (—QR₃⁺) including trialkylphosphonium or triarylphosphonium or mixed alkyl aryl phosphonium groups, and ternary sulfonium groups (—QR₂⁺). The solid phase can contain more than one kind of nucleic acid binding group as described herein. Solid phase materials containing ternary or quaternary onium groups—QR₂⁺ or —QR₃⁺ wherein the R groups are alkyl of at least four carbons are especially effective in binding nucleic acids, but alkyl groups of as little as one carbon are also useful as are aryl groups. Such solid phase materials retain the bound nucleic acid with great tenacity and resist removal or elution of the nucleic acid under most conditions used for elution known in the prior art. Known elution conditions of both low and high ionic strength are ineffective in removing bound nucleic acids. Unlike conventional anion-exchange resins containing DEAE and PEI groups, the ternary or quaternary onium solid phase materials remain positively charged regardless of the pH of the reaction medium.

In one aspect of the invention, there is provided a solid phase comprising a solid support portion comprising a matrix selected from silica, glass, insoluble synthetic polymers, and insoluble polysaccharides to which is attached on a surface a nucleic acid binding portion for attracting and binding nucleic acids, the nucleic acid binding portion (NAB) being linked by a cleavable linker portion to the solid support portion.

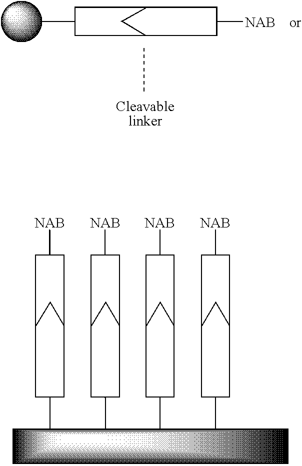

In one embodiment the NAB is a ternary onium group of the formula QR₂⁺X⁻ wherein Q is a S atom or a quaternary onium group QR₃⁺X⁻ wherein Q is a N or P atom, R is selected from alkyl, aralkyl and aryl groups and X is an anion. When Q is a nitrogen atom, the R groups will each contain from 4-20 carbon atoms. When Q is a sulfur or phosphorus atom, the R groups can have from 1-20 carbon atoms.

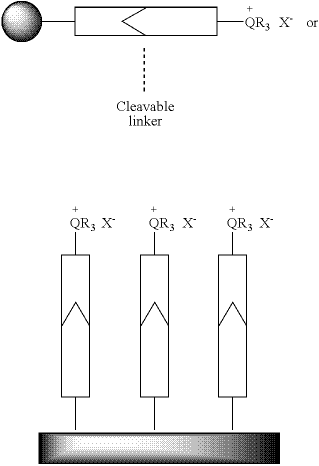

A preferred solid phase according to the present invention is derived from commercially available polystyrene type polymers such as those of the kind referred to as Merrifield resin (crosslinked). In these polymers a percentage of the styrene units contain a reactive group, typically a chloromethyl or hydroxymethyl group as a means of covalent attachment. Replacement of some of the chlorines by reaction with a sulfide (R₂S) or a tertiary amine or phosphine produces the solid phase materials of the invention. A polymer prepared in accordance with this definition can be depicted by the formula (1) below when all of the reactive chloromethyl groups have been converted to ternary or quaternary onium groups. It is not necessary for all such groups to be converted so that polymeric solid phases of the invention will often contain a mixture of the onium group and the chloromethyl group.

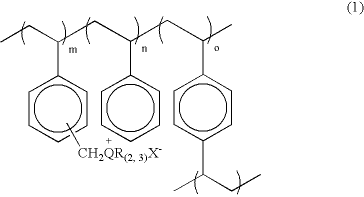

In the formula above, m, n, and o denote the mole percentage of each monomeric unit in the polymer and can take the values m from 0.1% to 100%, n from 0 to 99%, and o from 0 to 10%. More preferably m is from 1% to 20%, n is from 80 to 99%, and o is from 0 to 10%.

In another embodiment, a solid phase according to the present invention is derived from a commercially available crosslinked Merrifield resin having a percentage of the styrene units contain a reactive chloroacetyl or chloropropionyl group for covalent attachment. Ternary or quaternary onium polymers of the invention prepared from these starting polymers have the formula: 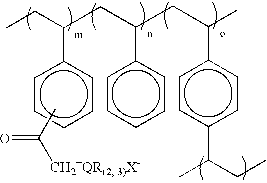
where Q, R, X, m, n, and o are as defined above.

Numerous other art-known polymeric resins can be used as the solid matrix in preparing solid phase materials of the invention. Polymeric resins are available from commercial suppliers such as Advanced ChemTech (Louisville, Ky.) and NovaBiochem. The resins are generally based on a crosslinked polymeric particle having a reactive functional group. Many suitable polymeric resins used in solid supported peptide synthesis as described in the Advanced ChemTech 2002 Catalog, pp. 105-140 are appropriate starting materials. Polymers having reactive NH₂, NH—NH₂, OH, SH, CHO, COOH, CO₂CH═CH₂, NCO, Cl, Br, SO₂CH═CH₂, SO₂Cl, SO₂NH₂, acylimidazole, oxime (C═N—OH), succinimide ester groups are each commercially available for use in preparation of polymeric solid phases of the invention. As is shown below in numerous examples it is sometimes necessary or desirable to provide a means of covalently joining a precursor polymer resin to the ternary or quaternary onium group. This will generally comprise a chain or ring group of 1-20 atoms selected from alkylene, arylene or aralkylene groups. The chain or ring can also contain O, S, or N atoms and carbonyl groups in the form of ketones, esters, thioesters, amides, urethanes, carbonates, xanthates, ureas, imines, oximes, sulfoxides and thioketones.

Solid phase materials of the invention having as the solid matrix a silica, glass or polysaccharide support will be functionalized by covalent attachment of a divalent group that links the nucleic acid binding group and the cleavable linker portion to the solid matrix. The divalent group will frequently be an organic group, either a low molecular weight group or a polymeric group. The divalent group can also be an organosilane. Suitable silanes useful to coat microparticle surfaces include p-aminopropyl-trimethoxysilane, N-2-amino-ethyl-3-aminopropyltrimethoxy-silane, (H₂NCH₂NHCH₂CH₂NHCH₂Si(OCH₃)₃, triethoxysilane and trimethoxysilane. Methods of preparing these microparticles are described in U.S. Pat. Nos. 4,628,037, 4,554,088, 4,672,040, 4,695,393 and 4,698,302, the teachings of which are hereby incorporated by reference. Silica particle materials having covalently bound organic linker groups are known and commercially available. One source describing numerous such materials is Silicycle (Quebec City, Canada). Silica particles bound via alkylene or other linkers to various reactive functional groups are described in a product catalog devoted to silica-based materials for solid phase synthesis. Representative functional groups depicted include amines, carbodiimide, carbonate, dichlorotriazine, isocyanate, maleimide, anhydride, carboxylic acid, carboxylic ester, thiol, thiourea, thiocyanate, sulfonyl chloride, sulfonic acid, and sulfonyl hydrazide groups. Any of these materials can serve to provide a solid matrix for attachment of a ternary or quaternary onium group as described above.

As used herein, magnetic microparticles are particles that can be attracted and manipulated by a magnetic field. The magnetic microparticles used in the method of the present invention comprise a magnetic metal oxide core, which is generally surrounded by an adsorptively or covalently bound layer to which a nucleic acid binding layer is covalently bound through selected coupling chemistries, thereby coating the surface of the microparticles with ternary sulfonium, quaternary ammonium, or quaternary phosphonium functional groups. The magnetic metal oxide core is preferably iron oxide, wherein iron is a mixture of Fe²⁺ and Fe³⁺. Magnetic microparticles comprising an iron oxide core, as described above, without a silane coat can also be used in the method of the present invention. Magnetic particles can also be formed as described in U.S. Pat. No. 4,654,267 by precipitating metal particles in the presence of a porous polymer to entrap the magnetically responsive metal particles. Magnetic metal oxides preparable thereby include Fe₃O₄, MnFe₂O₄, NiFe₂O₄, and CoFe₂O₄. Other magnetic particles can also be formed as described in U.S. Pat. No. 5,411,730 by precipitating metal oxide particles in the presence of a the oligosaccharide dextran to entrap the magnetically responsive metal particles. Yet another kind of magnetic particle is disclosed in the aforementioned U.S. Pat. No. 5,091,206. The particle comprises a polymeric core particle coated with a paramagnetic metal oxide particle layer and additional polymeric layers to shield the metal oxide layer and to provide a reactive coating. Preparation of magnetite containing chloromethylated Merrifield resin is described in a publication (Tetrahedron Lett., 40 (1999), 8137-8140).

Commercially available magnetic silica or magnetic polymeric particles can be used as the starting materials in preparing cleavable magnetic particles in accordance with the present invention. Suitable types of polymeric particles having surface carboxyl groups are known by the tradenames SeraMag™ (Seradyn) and BioMag™ (Polysciences and Bangs Laboratories). A suitable type of silica magnetic particles is known by the tradename MagneSil™ (Promega). Silica magnetic particles are also available from Chemicell GmbH (Berlin).

Other Cleavable Solid Supports

In another embodiment, there are provided solid phase materials comprising a solid phase matrix selected from silica or glass, insoluble synthetic polymers, and insoluble polysaccharides and having a cleavable linker group for attaching an onium group to the solid phase. The onium group is of the formula QR₂⁺X⁻ wherein Q is an S atom or QR₃⁺X⁻ wherein Q is an N or P atom, R is selected from alkyl having from 1-20 carbon atoms, aralkyl and aryl groups and X is an anion. The cleavable linker serves two functions, 1) to physically connect the matrix to the ternary or quaternary onium group, and 2) to provide a means of breaking the connection between the solid support matrix and the quaternary onium group to which nucleic acid is attracted, thereby liberating the bound nucleic acid from the solid phase matrix. The linker can be any grouping of atoms forming a divalent, trivalent or polyvalent group, provided that it contains a cleavable moiety which can be cleaved by a particular chemical, enzymatic agent or photochemical reaction. The cleaving agent or reaction must sufficiently preserve the nucleic acid during the process of breaking the cleavable link in order that the nucleic acid is useful for downstream processes.

Polymers are homopolymers or copolymers of one or more ethylenically unsaturated monomer units and can be crosslinked or non-crosslinked. Preferred polymers are polyolefins including polystyrene and the polyacrylic-type polymers. The latter comprise polymers of various substituted acrylic acids, amides and esters, wherein the acrylic monomer may or may not have alkyl substituents on the 2- or 3-carbon.

Numerous other art-known polymeric resins can be used as the solid matrix in preparing solid phase materials of the invention. Polymeric resins are available from commercial suppliers such as Advanced ChemTech (Louisville, Ky.). The resins are generally based on a crosslinked polymeric particle having a reactive functional group. Many suitable polymeric resins used in solid supported peptide synthesis as described in the Advanced ChemTech 2002 Catalog, pp. 105-140 are appropriate starting materials. Polymers having reactive NH₂, NH—NH₂, OH, SH, CHO, COOH, CO₂CH═CH₂, NCO, Cl, Br, SO₂CH═CH₂, SO₂Cl, SO₂NH₂, acylimidazole, oxime (C═N—OH), succinimide ester groups are each commercially available for use in preparation of polymeric solid phases of the invention.

As is shown below in numerous examples it is sometimes necessary or desirable to provide a means of covalently joining a precursor polymer resin to the cleavable linker portion or for joining the cleavable linker portion to quaternary onium group. In these cases the linker group may also comprise one or more connecting portions. The latter will generally comprise a chain or ring group of 1-20 atoms selected from alkylene, arylene or aralkylene groups. The chain or ring can also contain O, S, or N atoms and carbonyl groups in the form of ketones, esters, thioesters, amides, urethanes, carbonates, xanthates, ureas, imines, oximes, sulfoxides and thioketones.

The cleavable linker portion is preferably an organic group selected from straight chains, branched chains and rings and comprises from 1 to 100 atoms and more preferably from 1 to about 50 atoms. The atoms are preferably selected from C, H, B, N, O, S, Si, P, halogens and alkali metals. An exemplary linker group is a hydrolytically cleavable group which is cleaved by hydrolysis. Carboxylic esters and anhydrides, thioesters, carbonate esters, thiocarbonate esters, urethanes, imides, sulfonamides, and sulfonimides are representative as are sulfonate esters. Another exemplary class of linker groups are those groups which undergo reductive cleavage. One representative group is an organic group containing a disulfide (S—S) bond which is cleaved by thiols such as ethanethiol, mercaptoethanol, and DTT. Another representative group is an organic group containing a peroxide (O—O) bond. Peroxide bonds can be cleaved by thiols, amines and phosphines. 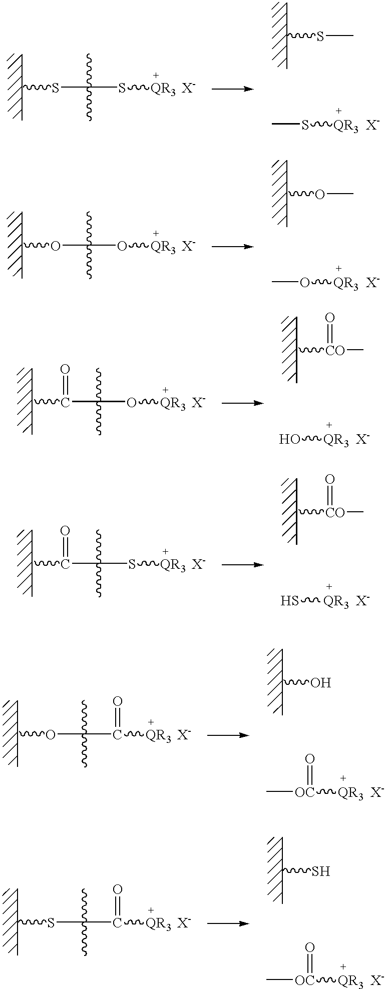
While many of the particular structure drawings represent only a quaternary onium group for convenience it should be understood that the analogous ternary sulfonium group is also meant to be represented as well.

Exemplary photochemically cleavable linker groups include nitro-substituted aromatic ethers and esters of the formula 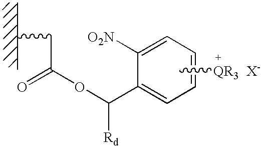
where R_{d} is H, alkyl or phenyl, and more particularly 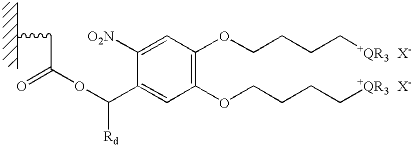
Ortho-nitrobenzyl esters are cleaved by ultraviolet light according to the well known reaction

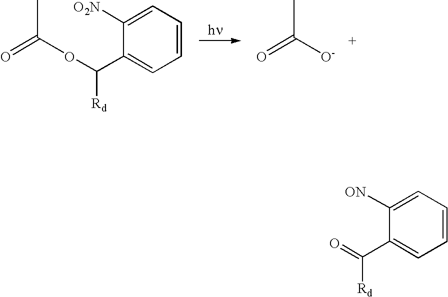

Exemplary enzymatically cleavable linker groups include esters which are cleaved by esterases and hydrolases, amides and peptides which are cleaved by proteases and peptidases, glycoside groups which are cleaved by glycosidases.

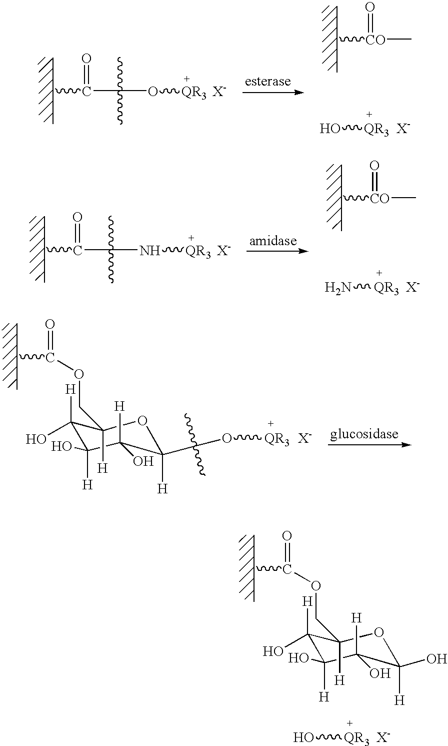

Solid phase materials having a linker group comprising a cleavable 1,2-dioxetane moiety are also within the scope of the inventive nucleic acid binding materials. Such materials contain a dioxetane moiety which can be triggered to fragment by a chemical or enzymatic agent. Removal of a protecting group to generate an oxyanion promotes decomposition of the dioxetane ring. Fragmentation occurs by cleavage of the peroxidic O—O bond as well as the C—C bond according to a well known process. 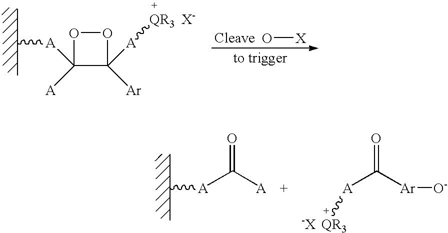
In the alternative, the linked onium group can be attached to the aryl group Ar as in: 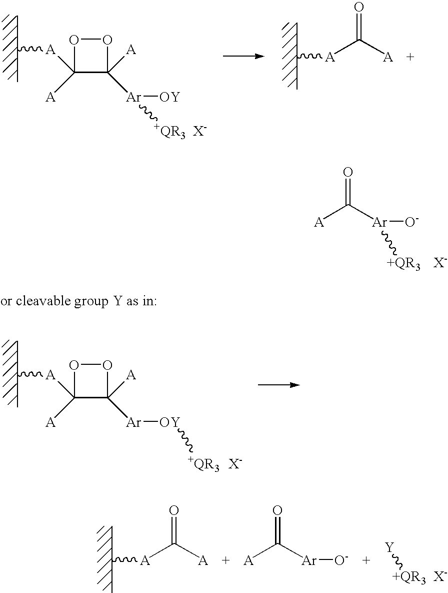
In a further alternative, the linkages to the solid phase and ternary or quaternary onium groups are reversed

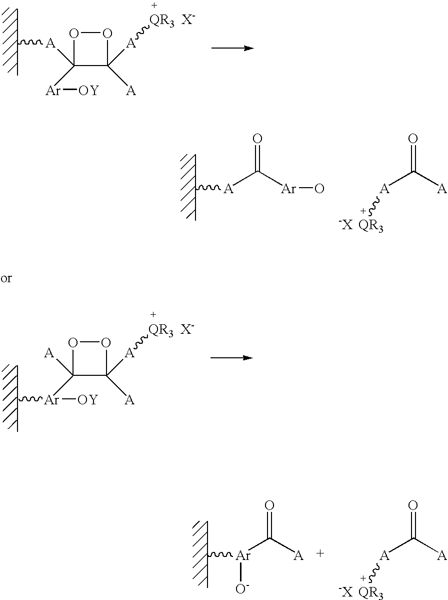

In the foregoing exemplary reactions for cleavage of the ternary or quaternary onium group from a solid phase, the groups A represent stabilizing substituents. Suitable groups are selected from alkyl, cycloalkyl, polycycloalkyl, polycycloalkenyl, aryl, aryloxy and alkoxy groups. Ar represents an aryl ring group. Preferred aryl ring groups are phenyl and naphthyl groups. The aryl ring can contain additional substituents, in particular halogens, alkoxy and amine groups. The Y group is a group or atom which is removable by a chemical agent or enzyme. Suitable OY groups include OH, OSiR³₃, wherein R³ is selected from alkyl and aryl groups, carboxyl groups, phosphate salts, sulfate salts, and glycoside groups. Numerous triggerable dioxetane structures are well known in the art and have been the subject of a large number of patents. The spiroadamantyl-stabilized dioxetanes disclosed in U.S. Pat. No. 5,707,559 are one example, others containing alkyl or cycloalkyl substituents as disclosed in U.S. Pat. No. 5,578,253 are also suitable. Many other variously substituted dioxetanes are described in the patent literature; any of these would also be suitable once linked to a solid phase and a nucleic acid binding group. Additional exemplary cleavable dioxetane structures are found in U.S. Pat. Nos. 6,036,892, 66,218,135, 6,228,653, 5,603,868, 6,107,036, 4,952,707, 6,140,495, 6,355,441 and 6,461,876.

A linking substituent from the aforementioned spiroadamantyl, alkyl or cycloalkyl groups is required to attach the dioxetane linker to either the solid phase or the ternary or quaternary onium group. Dioxetanes with linking groups are disclosed in U.S. Pat. No. 5,770,743 and illustrate the types of linkage chemistry available as connecting portions for covalent bonding of dioxetanes to the solid phase and the onium group. An exemplary cleavable dioxetane linker and its cleavage is depicted below. 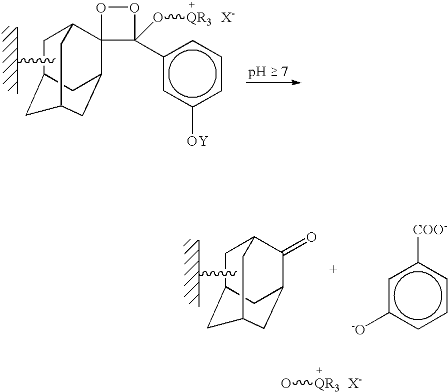
Removal of the protecting group Y triggers a fragmentation of the dioxetane ring and thereby separates the solid matrix and onium groups. Under alkaline reaction conditions the resulting aryl ester undergoes further hydrolysis.

Solid phase materials having a linker group comprising an electron-rich C—C double bond which can be converted to an unstable 1,2-dioxetane moiety are also within the scope of the inventive nucleic acid binding materials. At least one of the substituents (A′) on the double bond is attached to the double bond by means of an O, S, or N atom. Reaction of electron-rich double bonds with singlet oxygen produces an unstable 1,2-dioxetane group. The dioxetane ring spontaneously fragments at ambient temperatures, as described above to generate two carbonyl fragments.

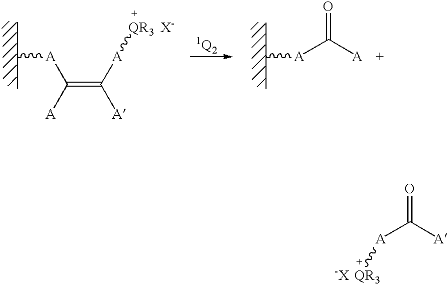

Another group of solid phase materials having a cleavable linker group have as the cleavable moiety a ketene dithioacetal as disclosed in PCT Publication WO 03/053934. Ketene dithioacetals undergo oxidative cleavage by enzymatic oxidation with a peroxidase enzyme and hydrogen peroxide.

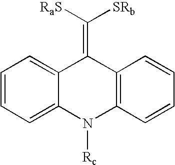

The cleavable moiety has the structure shown, including analogs having substitution on the acridan ring, wherein Rₐ and R_{b} are each organic groups containing from 1 to about 50 non-hydrogen atoms in addition to the necessary number of H atoms required to satisfy the valencies of the atoms in the group and wherein Rₐ and R_{b} can be joined together to form a ring. The groups Rₐ and R_{b} can contain from 1 to about 50 non-hydrogen atoms selected from C, N, O, S, P, Si and halogen atoms. R_{c} is an organic group containing from 1 to 50 non-hydrogen atoms selected from C, N, O, S, P, Si and halogen atoms in addition to the necessary number of H atoms required satisfy the valencies of the atoms in the group. More preferably R_{c} contains from 1 to 20 non-hydrogen atoms. The organic group R_{c} is preferably selected from the group consisting of alkyl, substituted alkyl, aryl, substituted aryl, aralkyl and substituted aralkyl groups. More preferred groups for R_{c} include substituted or unsubstituted C₁-C₄ alkyl groups, substituted or unsubstituted phenyl or naphthyl groups, and substituted or unsubstituted benzyl groups. When substituted, exemplary substituents include, without limitation, alkoxy, aryloxy, hydroxy, halogen, amino, substituted amino, carboxyl, carboalkoxy, carboxamide, cyano, sulfonate and phosphate groups. One preferred R_{c} group is an alkyl or heteroalkyl group substituted with at least one water-solubility conferring group.

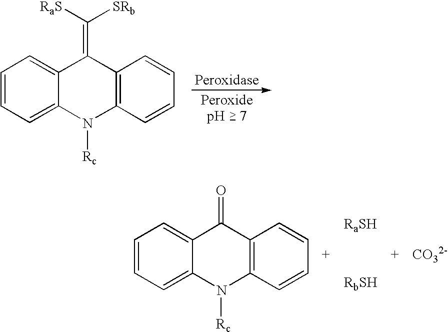

Solid phase materials having a ketene dithioacetal cleavable linker group can have any of the formulas: 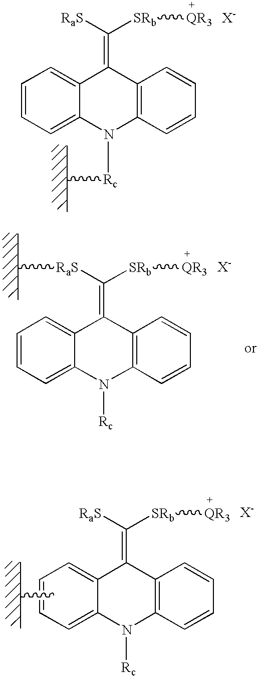
as well as the analogous structures where the order of attachment of the solid matrix and onium groups to the cleavable linker moiety is reversed from those shown.

Another group of solid phase materials having a cleavable linker group have as the cleavable moiety an alkylene group of at least one carbon atom bonded to a trialkyl or triarylphosphonium group. 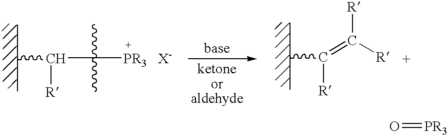
Materials of this group are cleavable by means of a Wittig reaction with a ketone or aldehyde. Reaction of a quaternary phosphonium compound with a strong base in an organic solvent deprotonates the carbon atom bonded to the phosphorus creating a phosphorus ylide. Reaction of the ylide with a carbonyl containing compound such as a ketone or aldehyde forms a double bond and the phosphine oxide. The link between the phosphonium group and the solid phase is broken in the process. Preferably the carbon atom joining the solid phase to the phosphorus atom (alpha carbon) is substituted in such a way that any attached protons are more acidic than any protons on the R groups on the phosphorus atom. Ylide formation and chain fragmentation are then directed to the correct site. In a preferred embodiment one of the other substituents on the carbon atom undergoing ylide formation is a phenyl group or a substituted phenyl group. When the quaternary phosphonium group is a triarylphosphonium group such as a triphenyl-phosphonium group, the requirement for enhanced acidity of the alpha proton is moot.

A further aspect of the invention comprises methods of isolating and purifying nucleic acids using the cleavable solid phase binding materials. In one embodiment there is provided a method of isolating a nucleic acid from a sample comprising:
a) providing a solid phase comprising:
   a solid support portion comprising a matrix selected from silica, glass, insoluble synthetic polymers, and insoluble polysaccharides,
   a nucleic acid binding portion for attracting and binding nucleic acids, and
   a cleavable linker portion;
b) combining the solid phase with the sample containing the nucleic acid to bind the nucleic acid to the solid phase;
c) separating the sample from the solid phase;
d) cleaving the cleavable linker; and
e) releasing the nucleic acid from the solid phase.

In a preferred embodiment the nucleic acid binding portion is a quaternary onium group of the formula QR₂⁺X⁻ or QR₃⁺X⁻ attached on a surface of the matrix wherein the quaternary onium group is selected from ternary sulfonium groups, quaternary ammonium, and phosphonium groups wherein R is selected from C₁-C₂₀ alkyl, aralkyl and aryl groups, and X is an anion.

The step of separating the sample from the solid phase can be accomplished by for example filtration, gravitational settling, decantation, magnetic separation, centrifugation, vacuum aspiration, overpressure of air or other gas as for example forcing a liquid through a porous membrane or filter mat. Components of the sample other than nucleic acids are removed in this step. To the extent that the removal of other components is not complete, additional washes can be performed to assist in their complete removal.

The step of cleaving the cleavable linker involves treatment of the solid phase having nucleic acid bound thereto with a cleaving agent for a period of time sufficient to break a covalent bond in the cleavable linker portion but not to destroy the nucleic acid. The choice of cleaving agent is determined by the nature of the cleavable linker. When the cleavable linker is a hydrolytically cleavable group, the cleaving agent is water or a lower alcohol or a mixture thereof. The cleaving agent preferably contains a base which when added to water raises the pH. Preferred bases are selected from hydroxide salts and alkoxide salts or contains a mineral acid or hydrogen peroxide. Exemplary bases include LiOH, NaOH, KOH, NH₄OH, NaOCH₃, KOCH₃, and KOt—Bu. When the cleavable linker is a reductively cleavable group such as a disulfide or peroxide group the cleaving agent is a reducing agent selected from thiols, amines and phosphines. Exemplary reducing agents include ethanethiol, 2-mercaptoethanol, dithiothreitol, trialkylamine and triphenylphosphine. Photochemically cleavable linker groups require the use of light as the cleaving agent, typically light in the ultraviolet region or the visible region. Enzymatically cleavable linker groups as described above are cleaved by enzymes selected from esterases, hydrolases, proteases, peptidases, peroxidases and glycosidases.

When the cleavable linker group is a triggerable dioxetane, the cleaving agent acts to cleave the O—Y bond in the triggering OY group as explained above. Cleaving the O—Y bond destabilizes the dioxetane ring group and leads to fragmentation of the dioxetane ring into two portions by rupture of the C—C and O—O bonds. When the OY group is OH the cleaving agent is an organic or inorganic base. When the OY group is OSiR³₃, wherein R³ is selected from alkyl and aryl groups, the cleaving agent is fluoride ion. When the OY group is joined to a carbonyl group, as in an ester, the cleaving agent is an esterase enzyme or is a chemical agent for hydrolyzing the ester. Such a chemical hydrolytic agent is selected from water or a lower alcohol or a mixture thereof. The cleaving agent preferably contains a base selected from hydroxide salts and alkoxide salts or contains a mineral acid or hydrogen peroxide. When the OY group is a phosphate salt the cleaving agent is a phosphatase enzyme. When the OY group is a sulfate salt the cleaving agent is a sulfatase enzyme. When the OY group is part of a glycoside group such as a glucoside or a galactoside the cleaving agent is the corresponding glycosidase enzyme.

When the cleavable linker is an electron-rich C—C double bond substituted with at least one O, S, or N atom, the cleaving agent is singlet oxygen. Reaction of the double bond group with singlet oxygen produces an unstable 1,2-dioxetane group which spontaneously fragments at ambient temperatures or above. The singlet oxygen can be generated by dye-sensitization or by thermolysis of triphenylphosphite ozonide or anthracene endoperoxides according to methods known in the art of singlet oxygenations.

When the cleavable linker is a ketene dithioacetal as described above, the cleaving agent is a peroxidase enzyme and hydrogen peroxide.

When the cleavable linker is an alkylene group of at least one carbon atom bonded to a trialkyl or triarylphosphonium group, cleaving is accomplished by a Wittig reaction with a ketone or aldehyde. The Wittig reaction is a well known reaction by which a quaternary phosphonium compound is deprotonated with a stong base in an organic solvent to create a phosphorus ylide. Reaction of the ylide with a carbonyl compound such as a ketone or aldehyde forms a double bond and the phosphine oxide. The link between the phosphonium group and the alpha carbon is broken as shown below. Preferably the alpha carbon is substituted with a group that renders an attached proton more acidic than any protons on the R groups on the phosphorus atom. Ylide formation and C—P bond fragmentation are then directed to the correct site. Preferred substituents on the alpha carbon are a phenyl group or a substituted phenyl group, an alkene group, an alkyne group or a carbonyl group. When the quaternary phosphonium group is a triarylphosphonium group such as a triphenylphosphonium group the requirement for enhanced acidity of the alpha proton is moot.

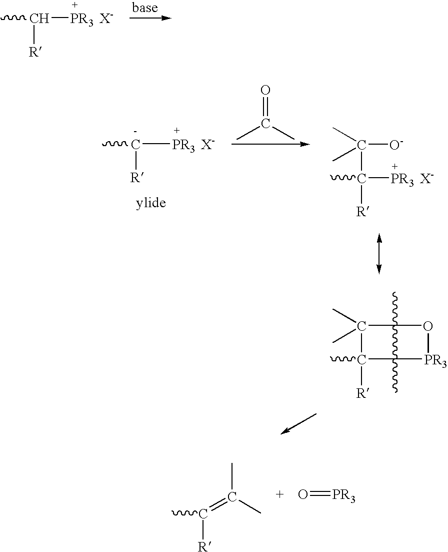

Preferred bases for forming the ylide are alkoxide salts and hydride salts, especially the alkali metal salts. Preferred carbonyl compounds for reaction with the ylide are aliphatic and aromatic aldehydes and aliphatic and aromatic ketones. More preferably the carbonyl compound does not have bulky groups to retard the rate of the reaction. Acetone is most preferred. Preferred solvents are aprotic organic solvent which can dissolve the reactants and do not consume the base including THF, diethyl ether, p-dioxane, DMF and DMSO.

The step of releasing the nucleic acid from the solid phase after cleavage comprises eluting with a solution which dissolves and sufficiently preserves the released nucleic acid. The solution can be a reagent composition comprising an aqueous buffer solution having a pH of 7-9, 0.1-3 M metal halide or acetate salt and a hydrophilic organic co-solvent at 1-50%. More preferably the hydrophilic organic solvent comprises from about 1-20%. Metal halide salts include alkali metal salts, alkaline earth salts. Preferred salts are sodium acetate, NaCl, KCl, and MgCl₂. Hydrophilic organic co-solvents are water soluble organic solvents and include methanol, ethanol, n-propanol, 2-propanol, t-butanol, ethylene glycol, propylene glycol, glycerol, 2-mercaptoethanol, dithiothreitol, furfuryl alcohol, 2,2,2-trifluoroethanol, acetone, THF, and p-dioxane. The step of releasing the captured nucleic acid can be subsequent to the cleaving step or concurrent with it. In the latter case the cleaving agent can also act as the elution solution.

The reagent for releasing the nucleic acid from the solid phase after cleavage can alternately be a strongly alkaline aqueous solution. Solutions of alkali metal hydroxides or ammonium hydroxide at a concentration of at least 10⁻⁴ M are effective in eluting nucleic acid from the cleaved solid phase.

The reagent for releasing the nucleic acid from the solid phase after cleavage can alternately be pure water or an alkaline buffered solution having a pH between about 8 and 10. Use of such alkaline buffers can be performed at temperatures up to 100° C. in order to increase the rate of cleavage. A buffer of moderately alkaline pH is useful particularly when the nucleic acid is RNA. Extended contact of RNA at very high pH, especially at high temperatures leads to its degradation.

The cleaving reaction and releasing (elution) steps can each be performed at room temperature, but any temperature above the freezing point of water and below the boiling point of water can be used. Elution temperature does not appear to be critical to the success of the present methods of isolating nucleic acids. Ambient temperature is preferred, but any temperature above the freezing point of water and below the boiling point of water can be used. Elevated temperatures may increase the rate of elution in some cases. The releasing or elution step can be performed once or can be repeated if necessary one or more times to increase the amount of nucleic acid released.

The cleaving reaction and elution steps can be performed as sequential steps using separate and distinct solutions to accomplish each step. Alternatively the cleaving and elution steps can be performed together in the same step. The latter, concurrent, method is preferred when the cleaving reaction conditions utilize reagents which are compatible with downstream uses of the eluted nucleic acid. Examples are cleaving reactions using moderately alkaline reaction buffers and even stronger alkaline solutions of sodium hydroxide. The former, sequential, method may be desirable in instance where the presence of reagents or solvents for the cleaving reaction are incompatible or undesirable with the nucleic acid. An example of this case is the Wittig release chemistry. Use of separate solutions for cleaving and elution is made possible when the cleaving reaction conditions do not substantially release the DNA into solution.

The method can further comprise a step of washing the solid phase having captured nucleic acid bound thereto with a wash solution to remove other components of the sample from the solid phase. These undesirable substances include enzymes, other types of proteins, polysaccharides, lower molecular weight substances, such as lipids and enzyme inhibitors. Nucleic acid captured on a solid phase of the invention by the above method can be used in captured form in a hybridization reaction to hybridize to labeled or unlabeled complementary nucleic acids. The hybridization reactions are useful in diagnostic tests for detecting the presence or amount of captured nucleic acid. The hybridization reactions are also useful in solid phase nucleic acid amplification processes.

Solid phase nucleic acid binding supports are also useful for binding and storing bound nucleic acid. Thus there is provided a method of capturing a nucleic acid from a sample comprising a method of isolating a nucleic acid from a sample comprising:
a) providing a solid phase comprising:
   a solid support portion comprising a matrix selected from silica, glass, insoluble synthetic polymers, and insoluble polysaccharides,
   a nucleic acid binding portion for attracting and binding nucleic acids, and
   a cleavable linker portion; and
b) combining the solid phase with the sample containing the nucleic acid to bind the nucleic acid to the solid phase.

In a preferred embodiment the nucleic acid binding portion is either a ternary onium group of the formula QR₂⁺X⁻ where Q is S and R is selected from C₁-C₂₀ alkyl, aralkyl and aryl groups or is a quaternary onium group of the formula QR₃+X⁻ attached on a surface of the matrix wherein the quaternary onium group is selected from quaternary ammonium groups wherein R is selected from C₄-C₂₀ alkyl, aralkyl and aryl groups, and quaternary phosphonium groups wherein R is selected from C₁-C₂₀ alkyl, aralkyl and aryl groups, and wherein X is an anion.

Release Without Cleavage

It has also been discovered that nucleic acid bound to solid supports of the present invention having as the cleavable linker an alkylene group of at least one carbon atom bonded to either a trialkyl or triarylphosphonium group, (i.e. those solid supports whereby cleavage is accomplished by a Wittig reaction with a ketone or aldehyde) or to a trialkylammonium group, can be made to release the nucleic acid by contact with certain reagent compositions. This result was unexpected since bound nucleic acid is not removed from these solid phase binding materials through contact with numerous other reagents and compositions known in the prior art to elute bound nucleic acids.

In another aspect of the invention then there is provided a method of isolating a nucleic acid from a sample comprising:
a) providing a solid phase comprising:
   a matrix selected from silica, glass, insoluble synthetic polymers, and insoluble polysaccharides, and
   an onium group attached on a surface of the matrix selected from a ternary sulfonium group of the formula QR₂⁺X⁻ where R is selected from C₁-C₂₀ alkyl, aralkyl and aryl groups, a quaternary ammonium group of the formula NR₃⁺X⁻ wherein the quaternary onium group wherein R is selected from C₄-C₂₀ alkyl, aralkyl and aryl groups, and a quaternary phosphonium group PR₃⁺X⁻ wherein R is selected from C₁-C₂₀ alkyl, aralkyl and aryl groups, and wherein X is an anion,
b) combining the solid phase with the sample containing the nucleic acid to bind the nucleic acid to the solid phase;
c) separating the sample from the solid phase; and
d) releasing the nucleic acid from the solid phase by contacting the solid phase with a reagent composition comprising an aqueous solution having a pH of 7-9, 0.1-3 M metal halide salt or acetate salt and a hydrophilic organic co-solvent at 1-50%.

The step of separating the sample from the solid phase can be accomplished by filtration, gravitational settling, decantation, magnetic separation, centrifugation, vacuum aspiration, overpressure of air or other gas to force a liquid through a porous membrane or filter mat, for example. Components of the sample other than nucleic acids are removed in this step. To the extent that the removal of other components is not complete, additional washes can be performed to assist in their complete removal.

Captured nucleic acid bound to the solid support is released from the solid support by elution with a reagent composition. The reagent composition comprises an aqueous solution having a pH of 7-9, 0.1-3 M metal halide salt or acetate salt and a hydrophilic organic co-solvent at 1-50%. More preferably the hydrophilic organic solvent comprises from about 1-20%. Metal halide salts include alkali metal salts and alkaline earth salts. Preferred salts are sodium acetate, NaCl, KCl, and MgCl₂. Hydrophilic organic co-solvents include methanol, ethanol, n-propanol, 2-propanol, t-butanol, 2-mercaptoethanol, dithiothreitol, furfuryl alcohol 2,2,2-trifluoroethanol, acetone, THF, and p-dioxane.

The elution composition advantageously permits use of the eluted nucleic acid directly in subsequent downstream processes without the need to evaporate the solvent or precipitate the nucleic acid before use.

Bound nucleic acid is surprisingly not removed from the above solid phase binding materials of the invention by washing with numerous reagents and compositions known in the prior art to elute bound nucleic acids. Eluents to which the solid phase materials were resistant include the list below. The listing includes high pH, high ionic strength and low ionic strength conditions.
deionized water H₂O
1 M phosphate buffer, pH 6.7
0.1% sodium dodecyl sulfate
0.1% sodium dodecyl phosphate
3 M potassium acetate, pH 5.5
TE (tris EDTA) buffer
50 mM tris, pH 8.5+1.25 M NaCl
0.3 M NaOH+1 M NaCl
1 M NaOH or
1 M NaOH+1 M H₂O₂.

When using a reagent composition as described above to elute nucleic acid, elution temperature does not appear to be critical to the success of the present methods of isolating nucleic acids. Ambient temperature is preferred, but any temperature above the freezing point of water and below the boiling point of water can be used. Elevated temperatures may increase the rate of elution in some cases.

In another aspect of the present invention there are provided novel reagent compositions for releasing or eluting bound nucleic acid molecules from the solid phase materials. Compositions of the invention comprise an aqueous solution having a pH of 7-9, 0.1-3 M metal halide salt or acetate salt and a hydrophilic organic co-solvent at 1-50%. More preferably the organic solvent comprises from about 1-20%. Hydrophilic organic co-solvents include methanol, ethanol, n-propanol, 2-propanol, t-butanol, 2-mercaptoethanol, dithiothreitol, furfuryl alcohol 2,2,2-trifluoroethanol, acetone, THF, and p-dioxane.

An important advantage of these reagent compositions is that they are compatible with many downstream molecular biology processes. Nucleic acid eluted into a reagent composition as described above can in many cases be used directly in a further process. Amplification reactions such as PCR, Ligation of Multiple Oligomers (LMO) described in U.S. Pat. No. 5,998,175, and LCR can employ such nucleic acid eluents. Nucleic acid isolated by conventional techniques, especially from bacterial cell culture or from blood samples, employ a precipitation step. Low molecular weight alcohols are added in high volume percent to precipitate nucleic acid from aqueous solutions. The precipitated materials must then be separated, collected and redissolved in a suitable medium before use. These steps can be obviated by elution of nucleic acid from solid phase binding materials of the present invention using the reagent compositions described above.

Samples from which nucleic acids can be isolated by the methods of the present invention comprise an aqueous solution containing one or more nucleic acids and, optionally, other substances. Representative examples include aqueous solutions of nucleic acids, amplification reaction products, and sequencing reaction products. Materials obtained from bacterial cultures, bodily fluids, blood and blood components, tissue extracts, plant materials, and environmental samples are likewise placed in an aqueous, preferably buffered, solution prior to use.

The methods of solid phase nucleic acid capture can be put to numerous uses. As shown in the particular examples below, both single stranded and double stranded nucleic acid can be captured and released. DNA, RNA, and PNA can be captured and released. A first use is in purification of plasmid DNA from bacterial culture. Plasmid DNA is used as a cloning vector to import a section of recombinant DNA containing a particular gene or gene fragment into a host for cloning.

A second use is in purification of amplification products from PCR or other amplification reactions. These reactions may be thermally cycled between alternating upper and lower temperatures, such as LMO or PCR, or they may be carried out at a single temperature, e.g., nucleic acid sequence-based amplification (NASBA). The reactions can use a variety of amplification reagents and enzymes, including DNA ligases, RNA polymerases and/or reverse transcriptases. Polynucleotide amplification reaction mixtures that may be purified using the methods of the invention include: ligation of multiple oligomers (LMO), self-sustained sequence replication (3SR), strand-displacement amplification (SDA), “branched chain” DNA amplification, ligase chain reaction (LCR), QB replicase amplification (QBR), ligation activated transcription (LAT), nucleic acid sequence-based amplification (NASBA), repair chain reaction (RCR), cycling probe reaction (CPR), and rolling circle amplification (RCA).

A third use is in sequencing reaction cleanup. Dideoxy terminated sequencing reactions produce ladders of polynucleotides resulting from extension of a primer with a mixture of dNTPs and one ddNTP in each of four reaction mixtures. The ddNTP in each is labeled, typically with a different fluorescent dye. Reaction mixtures contain excess dNTPs and labeled ddNTP, polymerase enzyme and cofactors such as ATP. It is desirable to remove the latter materials prior to sequence analysis.

A fourth use is in isolation of DNA from whole blood. DNA is extracted from leucocytes in a commonly used technique. Blood is typically treated to selectively lyse erythrocytes and after a precipitation or centrifugation step, the intact leucocytes are separately lysed to expose the nucleic acid content. Proteins are digested and the DNA obtained is isolated with a solid phase then used for determination of sequence polymorphism, sequence analysis, RFLP analysis, mutation detection or other types of diagnostic assay.

A fifth use is in isolating DNA from mixtures of DNA and RNA. Methods of the present invention involving strongly alkaline elution conditions, especially those using elevated temperatures, can degrade or destroy RNA present while leaving DNA intact. Methods involving strongly alkaline cleavage reactions will act similarly.

Additional uses include extraction of nucleic acid material from other samples—soil, plant, bacteria, and waste water and long term storage of nucleic acid materials for archival purposes.

Another advantage of the cleavable solid supports of the invention is that nucleic acids released from the support is contained in a solution which is compatible with many downstream molecular biology processes. Nucleic acid eluted into either a solution comprising the cleaving agent, when the solid phase comprises a cleavable linker, or into the reagent composition described above can, in many cases, be used directly in a further process. These processes include nucleic acid amplification reactions using either a polymerase or a ligase. Typical amplification reactions are PCR, Ligation of Multiple Oligomers (LMO) described in U.S. Pat. No. 5,998,175, and LCR. Use of solutions containing the released nucleic acid have been fund to be compatible with and not to substantially interfere with enzymatic and other reactions. Other downstream processes are described above and include nucleic acid hybridization assays, mutation detection and sequence analysis.

Thus a further aspect of the invention comprises methods of isolating and purifying nucleic acids using the cleavable solid phase binding materials. In one embodiment there is provided a method of isolating a nucleic acid from a sample comprising:
a) providing a solid phase comprising:
   a solid support portion comprising a matrix selected from silica, glass, insoluble synthetic polymers, and insoluble polysaccharides,
   a nucleic acid binding portion for attracting and binding nucleic acids, and
   a cleavable linker portion;
b) combining the solid phase with the sample containing the nucleic acid to bind the nucleic acid to the solid phase;
c) separating the sample from the solid phase;
d) cleaving the cleavable linker;
e) releasing the nucleic acid from the solid phase into a solution; and
f) further comprising using the solution containing the released nucleic acid directly in a downstream process.
   It is a preferred practice to use the solution containing the released nucleic acid directly in a nucleic acid amplification reaction whereby the amount of the nucleic acid or a segment thereof is amplified using a polymerase or ligase-mediated reaction.

The following examples are presented in order to more fully describe various aspects of the present invention. These examples do not limit the scope of the invention in any way.

### EXAMPLES

Structure drawings when present in the examples below are intended to illustrate only the cleavable linker portion of the solid phase materials. The drawings do not represent a full definition of the solid phase material.

### Example 1

### Synthesis of a Polystyrene Polymer Containing Tributylphosphonium Groups

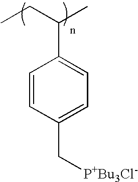

Merrifield peptide resin (Sigma, 1.1 meq/g, 20.0 g) which is a crosslinked chloromethylated polystyrene was stirred in 200 mL of CH₂Cl₂/DMF (50/50) under an argon pad. An excess of tributylphosphine (48.1 g, 10 equivalents) was added and the slurry was stirred at room temperature for 7 days. The slurry was filtered and the resulting solids were washed twice with 200 mL of CH₂Cl₂. The resin was dried under vacuum (21.5 g). Elemental Analysis: Found P 2.52%, Cl 3.08%; Expected P 2.79%, Cl 3.19%: P/Cl ratio is 0.94.

### Example 2

### Synthesis of a Polystyrene Polymer Containing Trioctylphosphonium Groups

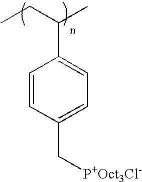

Merrifield peptide resin (Sigma, 1.1 meq/g, 20.0 g) was stirred in 200 mL of CH₂Cl₂/DMF (50/50) under an argon pad. An excess of trioctylphosphine (92.4 g, 10 equivalents) was added and the slurry was stirred at room temperature for 7 days. The slurry was filtered and the resulting solids were washed 3 times with 200 mL of CH₂Cl₂. The resin was dried under vacuum (21.3 g). Elemental Analysis: Found P 2.28%, Cl 2.77%; Expected P 2.77%, Cl 2.42%: P/Cl ratio is 0.94.

### Example 3

### Synthesis of a Polystyrene Polymer Containing Trimethylphosphonium Groups

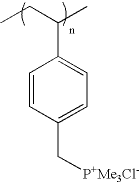

Merrifield peptide resin (ICN Biomedical, 1.6 meq/g, 5.0 g) was stirred in 50 mL of CH₂Cl₂ under an argon pad. A 1.0 M solution of trimethyl phosphine in THF (Aldrich, 12 mL) was added and the slurry was stirred at room temperature for 7 days. An additional 100 mL of CH₂Cl₂ and 1.2 mL of the 1.0 M solution of trimethyl phosphine in THF was added and the slurry was stirred for 3 days. The slurry was filtered and the resulting solids were washed with 125 mL of CH₂Cl₂ followed by 375 mL of methanol. The resin was dried under vacuum (5 g). The resin was ground to a fine powder prior to use.

### Example 4

### Synthesis of a Polystyrene Polymer Containing Triphenylphosphonium Groups

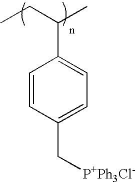

Merrifield peptide resin (ICN Biomedical, 1.6 meq/g, 5.0 g) was stirred in 40 mL of CH₂Cl₂ under an argon pad. Triphenyl phosphine (Aldrich, 3.2 g) was added and the slurry was stirred at room temperature for 5 days. The slurry was filtered and the resulting solids were washed sequentially with CH₂Cl₂, MeOH, and CH₂Cl₂. The resin was dried under vacuum (5.4 g).

### Example 5

### Synthesis of a Polystyrene Polymer Containing Tributylammonium Groups

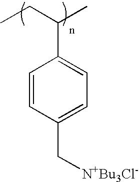

Merrifield peptide resin (Aldrich, 1.43 meq/g, 25.1 g) was stirred in 150 mL of CH₂Cl₂ under an argon pad. An excess of tributyl amine (25.6 g, 4 equivalents) was added and the slurry was stirred at room temperature for 8 days. The slurry was filtered and the resulting solids were washed twice with 250 mL of CH₂Cl₂. The resin was dried under vacuum (28.9 g). Elemental Analysis: Found N 1.18%, Cl 3.40%; Expected N 1.58%, Cl 4.01%: N/Cl ratio is 0.88.

### Example 6

### Synthesis of a Polystyrene Polymer Containing 2-(tributylphosphonium)acetyl Groups

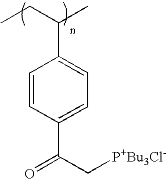

Chloroacetyl polystyrene beads (Advanced Chemtech, 3.0 g, 3.4 meq/g) was added to a solution of tributylphosphine (4.1 g, 2 equivalents) in 50 mL of CH₂Cl₂ under an argon pad. The slurry was stirred for one week. The slurry was filtered and the resulting solids were washed sequentially with CH₂Cl₂ (4×25 mL), MeOH (2×25 mL), and acetone (4×25 mL). The resin was then air dried.

### Example 7

### Synthesis of Magnetic Particle Having a Polymeric layer containing Polyvinylbenzyltributyl-Phosphonium Groups

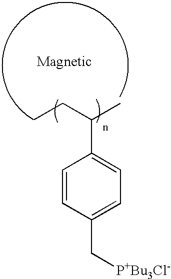

Magnetic Merrifield peptide resin (Chemicell, SiMag Chloromethyl, 100 mg) was added to 2 mL of CH₂Cl₂ in a glass vial. Tributylphosphine (80 μL) was added and the slurry was shaken at room temperature for 3 days. A magnet was placed under the vial and the supernatant was removed with a pipet. The solids were washed four times with 2 mL of CH₂Cl₂ (the washes were also removed by the magnet/pipet procedure). The resin was air dried (93 mg).

### Example 8

### Br

### Synthesis of Polymethacrylate Polymer Containing Tributylphosphonium Groups and Bromide Anion

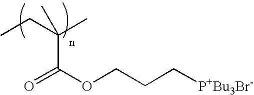

Polymethacrylic acid resin was refluxed with 35 mL of SOCl₂ for 4 h to form the acid chloride. Polymethacryloyl chloride resin (4.8 g) and triethylamine (11.1 g) were stirred in 100 mL of CH₂Cl₂ in an ice water bath under argon. 3-Bromopropanol (9.0 g) was added and the ice water bath was removed. The slurry was stirred overnight at room temperature. The slurry was filtered and the resin was washed 3 times with 40 mL of CH₂Cl₂. The resin was air dried (8.7 g).

The resin (8.5 g) was resuspended and stirred in 100 mL of CH₂Cl₂ under argon. Tributyl phosphine (16.2 g) was added and the slurry stirred for 7 days. The slurry was filtered and the resin was washed 3 times with 100 mL of CH₂Cl₂. The resin was then air dried (5.0 g).

### Example 8

### Cl

### Synthesis of Polymethacrylate Polymer Containing Tributylphosphonium Groups and Chloride Anion

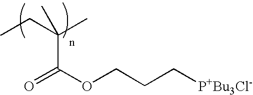

Polymethacryloyl chloride resin (12.2 g) and triethylamine (23.2 g) were stirred in 100 mL of CH₂Cl₂ in an ice water bath under argon. 3-Chloropropanol (12.8 g) was added and the ice water bath was removed. The slurry was stirred overnight at room temperature. The slurry was filtered and the resin was washed 3 times with 100 mL of CH₂Cl₂. The resin was air dried (12.8 g).

The resin (12.8 g) was resuspended and stirred in 100 mL of CH₂Cl₂ under argon. Tributyl phosphine (27.8 g) was added and the slurry stirred for 7 days. The slurry was filtered and the resin was washed with 2×100 mL of CH₂Cl₂ and 2×100 mL of MeOH. The resin was then air dried (9.8 g).

### Example 8

### S

### Synthesis of Polymethacrylate Polymer Containing Tributylphosphonium Groups and Alkylthioester Linkage

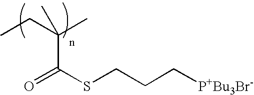

Polymethacryloyl chloride resin (3.6 g) and triethylamine (8.9 g) were stirred in 20 mL of CH₂Cl₂ in an ice water bath under argon. 3-Mercapto-1-propanol (5.8 g), diluted in 20 mL of CH₂Cl₂₁ was added and the ice water bath was removed. The slurry was stirred overnight at room temperature. The slurry was filtered and the resin was washed with CH₂Cl₂₁ water, and methanol. The resin was air dried (3.5 g).

The resin (4.3 g) was resuspended and stirred in 100 mL of dry acetonitrile under argon. Carbon tetrabromide (14.9 g) and triphenyl phosphine (11.8 g) were added. The mixture was refluxed for 5 hours. The slurry was filtered and the resin was washed with 125 mL of acetonitrile, 250 mL of MeOH, and 250 mL of CH₂Cl₂. The resin was then air dried (4.2 g).

The resin (4.2 g) was resuspended and stirred in 40 mL of CH₂Cl₂ under argon. Tributyl phosphine (6.7 g) was added and the slurry stirred for 8 days. The slurry was filtered and the resin was washed with 90 mL of CH₂Cl₂ followed by 50 mL of MeOH. The resin was then air dried (4.0 g).

### Example 9

### Synthesis of Polyvinylbenzyl Polymer Containing Tributylphosphonium Groups and Ester Linkage

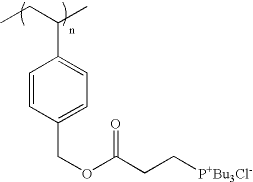

Polystyrene hydroxymethyl acrylate resin (5.0 g) was stirred in 50 mL of acetonitrile in an ice water bath under argon. Tributyl phosphine (2.1 g) and 4.0 M HCl (2.5 mL) were stirred under argon for 15 minutes. This solution was added in 4 equal portions to the resin slurry over 1 hour. The ice water bath was removed and the slurry was stirred at room temperature for 3 hours. The resin was filtered and washed with 50 mL of acetonitrile followed by two 50-mL portions of CH₂Cl₂. The resin was then air dried (6.24 g).

### Example 10

### Synthesis of Polyvinylbenzyl Polymer Containing Tributylphosphonium Groups and Ester Linkage

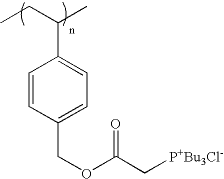

Hydroxymethylated polystyrene (Aldrich, 2.0 meq/g, 5.0 g) and triethylamine (2.3 g) were stirred in 100 mL of CH₂Cl₂ in an ice water bath under argon. Chloroacetyl chloride (1.9 g) was added and the ice water bath was removed. The slurry was stirred overnight at room temperature. The slurry was filtered and the resin was washed 3 times with 40 mL of CH₂Cl₂. The resin was air dried (5.8 g).

The resin (5.8 g) was resuspended and stirred in 100 mL of CH₂Cl₂ under argon. Tributyl phosphine (3.2 g) was added and the slurry stirred for 7 days. The slurry was filtered and the resin was washed 2 times with 100 mL of CH₂Cl₂. The resin was then air dried (5.9 g).

### Example 11

### Synthesis of Polymethacrylate Polymer Containing Tributylphosphonium Groups and Two Ester Linkages

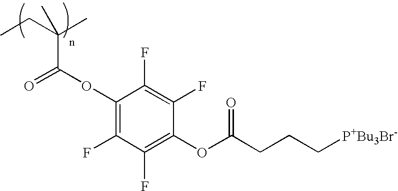

Polymethacryloyl chloride resin and pyridine were stirred in 50 mL of CH₂Cl₂ in an ice water bath under argon. Tetrafluorohydroquinone (2.7 g) was added and the ice water bath was removed. The slurry was stirred for 43 hours at room temperature. The slurry was filtered and the resin was washed sequentially with CH₂Cl₂₁ water, MeOH, and CH₂Cl₂. The resin was air dried (1.3 g).

The resin and triethylamine (662 mg) were stirred in 30 mL of CH₂CO₂ in an ice water bath under argon. 4-Bromobutyryl chloride (1.12 g) was added and the ice water bath was removed. The slurry was stirred for 2 days at room temperature. The slurry was filtered and the resin was washed sequentially with CH₂Cl₂, water, MeOH, and CH₂Cl₂. The resin was air dried (1.3 g).

The resin was resuspended and stirred in 18 mL of CH₂Cl₂ under argon. Tributyl phosphine (4.7 g) was added and the slurry stirred for 10 days. The slurry was filtered and the resin was washed sequentially with CH₂Cl₂₁ MeOH, and CH₂Cl₂. The resin was then air dried (1.3 g).

### Example 12

### Synthesis of Photocleavable Polymethacrylate Polymer Containing Tributylphosphonium Groups and Ester Linkage

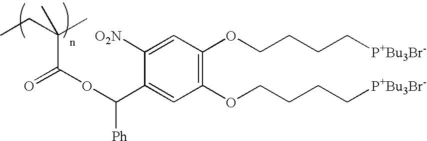

Polymethacryloyl chloride resin (2.0 g) and triethylamine (4.2 g) were stirred in 25 mL of CH₂Cl₂ in an ice water bath under argon. [4,5-Bis(4-bromo-1-butoxy)-2-nitrophenyl)]-phenyl methanol (16.7 g) was diluted in 100 mL of CH₂Cl₂ and added. The ice water bath was removed and the slurry was stirred overnight at room temperature. The slurry was filtered and the resin was washed 2 times with 100 mL of CH₂Cl₂. The resin was air dried (2.5 g).

The resin (2.5 g) was resuspended and stirred in 50 mL of CH₂Cl₂ under argon. Tributyl phosphine (4.0 g) was added and the slurry stirred for 7 days. The slurry was filtered and the resin was washed 2 times with 50 mL of CH₂Cl₂. The resin was then air dried (2.4 g).

### Example 13

### Synthesis of Polymethacrylate Polymer Containing Tributylphosphonium Groups and Arylthioester Linkage

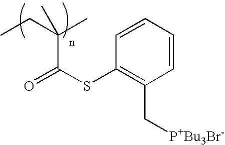

Polymethacryloyl chloride resin (2.7 g) and triethylamine (8.6 g) were stirred in 25 mL of CH₂Cl₂ in an ice water bath under argon. 2-Mercaptobenzyl alcohol (5.0 g), diluted in 20 mL of CH₂Cl₂, was added and the ice water bath was removed. The slurry was stirred for 2 days at room temperature. The slurry was diluted with 50 mL of CH₂Cl₂ and centrifuged for 10 minutes at 6000 rpm. The supernatant was discarded. The resin was washed 3 times with 100 mL of MeOH (each wash was centrifuged for 10 minutes at 6000 rpm). After the last wash, the resin was filtered and air dried (4.2 g).

The resin (3.4 g) was resuspended and stirred in 100 mL of dry acetonitrile under argon. Carbon tetrabromide (10.2 g) and triphenyl phosphine (8.0 g) were added. The mixture was refluxed for 4 hours. The slurry was filtered and the resin was washed with 125 mL of acetonitrile, 250 mL of MeOH, and 250 mL of CH₂Cl₂. The resin was then air dried (2.8 g).

The resin (2.8 g) was resuspended and stirred in 40 mL of CH₂Cl₂ under argon. Tributyl phosphine (4.0 g) was added and the slurry stirred for 8 days. The slurry was filtered and the resin was washed with 50 mL of CH₂Cl₂ followed by 125 mL of MeOH. The resin was then air dried (2.7 g).

### Example 14

### Synthesis of Polymethacrylate Polymer Containing Trimethylphosphonium Groups and Arylthioester Linkage

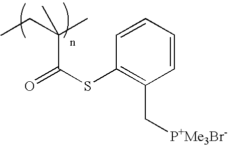

Polymethacryloyl chloride resin (5.1 g) and triethylamine (12.3 g) were stirred in 100 mL of CH₂Cl₂ under argon. 2-Mercaptobenzyl alcohol (9.3 g) was added and the slurry stirred for 5 days at room temperature. The slurry was filtered and the resin was washed with 300 mL of CH₂Cl₂, 500 mL of water, and 200 mL of MeOH. The resin was air dried (5.8 g).

The resin (4.8 g) was resuspended and stirred in 100 mL of dry acetonitrile under argon. Carbon tetrabromide (14.3 g) and triphenyl phosphine (11.3 g) were added. The mixture was refluxed for 4 hours. The slurry was filtered and the resin was washed with 100 mL of acetonitrile, 200 mL of CH₂Cl₂, 200 mL of MeOH, and 200 mL of CH₂Cl₂. The resin was then air dried (4.8 g).

The resin (1.04 g) was resuspended and stirred in 30 mL of CH₂Cl₂ under argon. A 1.0 M solution of trimethyl phosphine in THF (7.3 mL) was added and the slurry stirred for 10 days. The slurry was filtered and the resin was washed with 100 mL of CH₂Cl₂₁ 100 mL of THF, 50 mL of MeOH, and 100 mL of CH₂Cl₂. The resin was then air dried (1.10 g).

### Example 15

### Synthesis of Polymethacrylate Polymer Containing Trioctylphosphonium Groups and Arylthioester Linkage

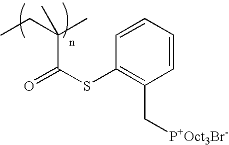

Polymethacryloyl chloride resin (5.1 g) and triethylamine (12.3 g) were stirred in 100 mL of CH₂Cl₂ under argon. 2-Mercaptobenzyl alcohol (9.3 g) was added and the slurry stirred for 5 days at room temperature. The slurry was filtered and the resin was washed with 300 mL of CH₂Cl₂, 500 mL of water, and 200 mL of MeOH. The resin was air dried (5.8 g).

The resin (4.8 g) was resuspended and stirred in 100 mL of dry acetonitrile under argon. Carbon tetrabromide (14.3 g) and triphenylphosphine (11.3 g) were added. The mixture was refluxed for 4 hours. The slurry was filtered and the resin was washed with 100 mL of acetonitrile, 200 mL of CH₂Cl₂, 200 mL of MeOH, and 200 mL of CH₂Cl₂. The resin was then air dried (4.8 g).

The resin (1.68 g) was resuspended and stirred in 30 mL of CH₂Cl₂ under argon. Trioctylphosphine (4.4 g) was added and the slurry stirred for 10 days. The slurry was filtered and the resin was washed with 100 mL of CH₂Cl₂, 100 mL of THF, 50 mL of MeOH, and 100 mL of CH₂Cl₂. The resin was then air dried (1.67 g).

### Example 16

### Synthesis of Magnetic Silica Particles Functionalized with Polymethacrylate Linker and Containing Tributylphosphonium Groups and Arylthioester Linkage

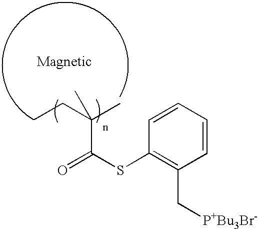

Magnetic carboxylic acid-functionalized silica particles (Chemicell, SiMAG-TCL, 1.0 meq/g, 0.6 g) were placed in 6 mL of thionyl chloride and refluxed for 3 hours. The excess thionyl chloride was removed under reduced pressure. The resin was resuspended in 40 mL of CH₂Cl₂ in an ice water bath under argon. Triethylamine (1.2 g) was added. 2-Mercaptobenzyl alcohol (0.7 g) was added and the ice water bath was removed. The slurry was shaken overnight at room temperature. The slurry was filtered and the resin was centrifuged twice with 35 mL of MeOH at 5000 rpm for 10 minutes. The supernatants were discarded. The orange-yellow resin was air dried (335 mg).

The resin (335 mg) was resuspended in 45 mL of dry acetonitrile under argon. Carbon tetrabromide (2.0 g) and triphenylphosphine (1.6 g) were added. The mixture was refluxed for 3 hours. The resin was centrifuged at 5000 rpm for 10 minutes and the supernatant was discarded. The resin was centrifuged twice with 50 mL of acetonitrile at 5000 rpm for 10 minutes and the supernatants were discarded. The resin was then air dried (328 mg).

The resin (328 mg) was resuspended in 40 mL of CH₂Cl₂ under argon. Tributylphosphine (280 mg) was added and the slurry shaken for 10 days. The magnetic resin settled by placing a magnet on the exterior of the flask and the supernatant was decanted. The resin was washed 3 times with 30 mL of CH₂Cl₂ followed with 3 washes of 25 mL of MeOH. The resin was then air dried (328 mg).

### Example 17

### Synthesis of Magnetic Polymeric Methacrylate Particles Containing Tributylphosphonium Groups and Arylthioester Linkage

Sera-Mag™ Magnetic Carboxylate Microparticles (Seradyn) were used to form cleavable magnetic particles. The Sera-Mag particles comprise a polystyrene-acrylic acid polymer core surrounded by a magnetite coating encapsulated with proprietary polymers. Carboxylate groups are accessible on the surface. Particles (0.52 meq/g, 0.50 g) were suspended in 15 mL of water and 25 mL of 0.1 M MES buffer (pH 4.0). The reaction mixture was sonicated for 5 minutes prior to the addition of 126 mg of EDC (1-[3-(dimethylamino)propyl]-3-ethyl carbodiimide hydrochloride) and 110 mg of 2-mercaptobenzyl alcohol. The reaction mixture was shaken for 7 days. The reaction mixture was filtered. The resin was washed with 50 mL of water and 100 mL of MeOH. The resin was air dried (0.53 g).

The resin (0.53 g) was resuspended in 20 mL of dry acetonitrile under argon. Carbon tetrabromide (174 mg) and triphenyl phosphine (138 mg) were added. The mixture was sonicated at 65° C. for 5 hours. The reaction mixture was placed on a large magnet and the supernatant was decanted. The resin was washed 4 times with acetonitrile, the resin was precipitated by a magnet, and the washes were discarded. The resin was resuspended in MeOH and shaken overnight. The resin was washed 4 times with MeOH, the resin was precipitated by a magnet, and the washes were discarded. The resin was then air dried (0.52 g).

The resin (0.52 g) was resuspended in 10 mL of acetonitrile. Tributylphosphine (106 mg) was added and the reaction shaken for 7 days. The magnetic resin was precipitated by a magnet and the supernatant was decanted. The resin was washed 4 times with acetonitrile and 4 times with MeOH. The resin was then air dried (0.51 g).

### Example 18

### Synthesis of Polymethacrylate Polymer Containing Tributylphosphonium Groups and Arylthioester Linkage

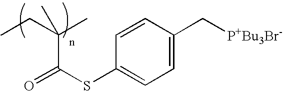

Polymethacryloyl chloride resin (0.6 g) and triethylamine (1.5 g) were stirred in 30 mL of CH₂Cl₂ in an ice water bath under argon. 4-Mercaptobenzyl alcohol (1.0 g), diluted in 20 mL of CH₂Cl₂, was added and the ice water bath was removed. The slurry was stirred for 2 days at room temperature. The slurry was filtered and washed with 50 mL of CH₂Cl₂, 100 mL of water, 50 mL of MeOH, and 25 mL of CH₂Cl₂. The resin was air dried (0.7 g).

The resin (0.6 g) was resuspended and stirred in 20 mL of dry acetonitrile under argon. Carbon tetrabromide (1.8 g) and triphenylphosphine (1.4 g) were added. The mixture was refluxed for 3 hours. The slurry was filtered and the resin was washed with acetonitrile, MeOH, and CH₂Cl₂. The resin was then air dried (0.6 g).

The resin (0.6 g) was resuspended and stirred in 15 mL of CH₂Cl₂ under argon. Tributylphosphine (0.85 g) was added and the slurry stirred for 6 days. The slurry was filtered and the resin was washed with 75 mL of CH₂Cl₂ followed by 150 mL of MeOH. The resin was then air dried (0.6 g).

### Example 19

### Synthesis of Polymethacrylate Polymer Containing Tributylphosphonium Groups and Arylthioester Linkage

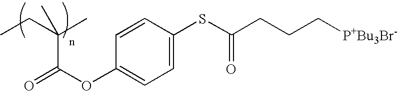

Polymethacryloyl chloride resin (0.71 g) and triethylamine (2.2 g) were stirred in 100 mL of CH₂Cl₂ under argon. 4-Hydroxyphenyl 4-bromothiobutyrate (2.55 g) was added and the slurry was stirred for 5 days at room temperature. The slurry was filtered and washed with CH₂Cl₂ and hexanes. The resin was air dried (0.85 g).

The resin (0.85 g) was resuspended and stirred in 20 mL of CH₂Cl₂ under argon. Tributylphosphine (2.7 g) was added and the slurry stirred for 3 days. The slurry was filtered and the resin was washed with CH₂Cl₂ and hexanes. The resin was then air dried.

### Example 20

### Synthesis of Polymethacrylate Polymer Containing Tributylphosphonium Groups and Arylthioester Linkage

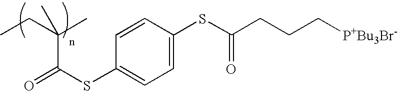

Polymethacryloyl chloride resin (1.0 g) and pyridine (1.9 mL) were stirred in 20 mL of CH₂Cl₂ under argon. 1,4-Benzene dithiol (1.85 g) was added and the slurry was stirred overnight at room temperature. The slurry was filtered and washed with CH₂Cl₂ and hexanes. The resin was air dried (1.08 g).

The resin (1.08 g) and triethylamine (3.0 mL) were stirred in 20 mL of CH₂Cl₂ under argon. 4-Bromobutyryl chloride (1.8 mL) was added and the reaction mixture was stirred for 2 days. The slurry was filtered and washed with CH₂Cl₂. The resin was air dried (1.10 g).

The resin (1.10 g) was resuspended and stirred in 30 mL of CH₂Cl₂ under argon. Tributylphosphine (4.0 g) was added and the slurry stirred for 5 days. The slurry was filtered and the resin was washed with CH₂Cl₂. The resin was then air dried (1.0 g).

### Example 21

### Synthesis of Crosslinked Polystyrene Polyethylene Glycol Succinate Copolymer Containing Tributylphosphonium Groups

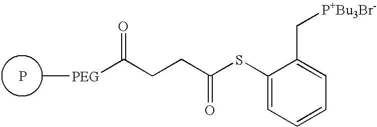

TentaGel S COOH beads (Advanced Chemtech, 3.0 g), a crosslinked polystyrene polyethylene glycol succinate copolymer, were refluxed in 30 mL of thionyl chloride for 90 minutes. The residual thionyl chloride was removed under reduced pressure. The resin was resuspended in 30 mL of chloroform and reconcentrated.

The resin and triethylamine (0.14 g) were stirred in 60 mL of CH₂Cl₂ in an ice water bath under argon. 2-Mercapto-benzyl alcohol (0.11 g) was added and the ice water bath was removed. The slurry was stirred for 2 days at room temperature. The slurry was filtered and the resin was washed with CH₂Cl₂, water, MeOH, and CH₂Cl₂. The resin was filtered and air dried (2.9 g).

The resin (2.8 g) was resuspended and stirred in 60 mL of dry acetonitrile under argon. Carbon tetrabromide (0.36 g) and triphenylphosphine (0.29 g) were added. The mixture was refluxed for 4 hours. The slurry was filtered and the resin was washed with acetonitrile, MeOH, and CH₂Cl₂. The resin was then air dried (2.8 g).

The resin (2.7 g) was resuspended and stirred in 50 mL of CH₂Cl₂ under argon. Tributylphosphine (0.21 g) was added and the slurry stirred for 6 days. The slurry was filtered and the resin was washed with 50 mL of CH₂Cl₂ followed by 175 mL of MeOH. The resin was then air dried (2.8 g).

### Example 22

### Synthesis of Controlled Pore Glass Beads Containing Succinate-Linked Tributylphosphonium Groups and a Thioester Linkage

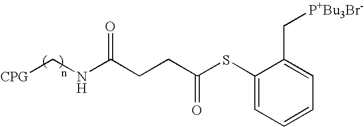

Millipore LCAA glass (1.0 g, 38.5 μmole/gram) was suspended in 10 mL of dry pyridine. Succinic anhydride (40 mg) was added and the reaction mixture was shaken at room temperature for 4 days. The reaction mixture was diluted with 20 mL of MeOH and the mixture was filtered. The solids were washed 5 times with 20 mL of MeOH and 5 times with 20 mL of CH₂Cl₂. The solids were air dried (1.0 g).

The solids (0.50 g) were suspended in 10 mL of dry CH₂Cl₂. Dicyclohexylcarbodiimide (10 mg) and 2-mercaptobenzyl alcohol were added and the reaction mixture was shaken at room temperature for 6 days. The reaction mixture was diluted with CH₂Cl₂ and the mixture was filtered. The solids were washed 3 times with MeOH and 3 times with CH₂Cl₂. The solids were air dried (0.50 g).

The solids (400 mg) were resuspended in 10 mL of dry acetonitrile under argon. Carbon tetrabromide (14 mg) and triphenylphosphine (11 mg) were added. The mixture was refluxed for 3 hours. The mixture was filtered and the solid was washed 5 times with 50 mL of MeOH and 5 times with 50 mL of CH₂Cl₂. The solids were air dried (360 mg).

The solid (300 mg) was resuspended in 10 mL of CH₂Cl₂ under argon. Tributylphosphine (5 drops) was added and the reaction mixture was shaken for 5 days. The reaction mixture was diluted with CH₂Cl₂ and filtered. The solid was washed 5 times with 50 mL of CH₂Cl₂ and air dried (300 mg).

### Example 23

### Synthesis of Polyvinylbenzyl Polymer Containing Acridinium Ester Groups

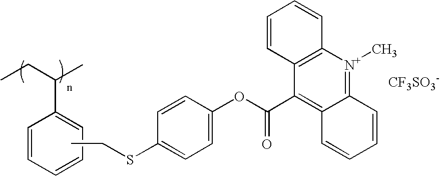

Acridine 9-carboxylic acid chloride, 1.25 g) and triethylamine (1.3 g) were stirred in 40 mL of CH₂Cl₂ in an ice water bath under argon. Hydroxythiophenol resin (Polymer Laboratories, 1.67 meq/g, 3.0 g) was added and the ice water bath was removed. The slurry was stirred overnight at room temperature. The slurry was filtered and the resin was washed 3 times with 200 mL of CH₂Cl₂. The resin was air dried (4.4 g).

The resin (4.3 g) was stirred in 40 mL of CH₂Cl₂ under argon. Methyl triflate (6.1 g) was added and the reaction mixture was stirred for 2 days. The slurry was filtered and the resin was washed with 200 mL of CH₂Cl₂ and 1 L of MeOH. The resin was vacuum-dried (4.7 g).

### Example 24

### Synthesis of Polyvinylbenzyl Polymer Containing Acridan Ketene Dithioacetal Groups

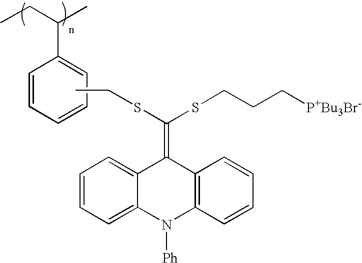

N-Phenyl acridan (0.62 g) was stirred in 20 mL of anhydrous THF at −78° C. under argon. Butyl lithium (2.5 M in hexanes, 0.93 mL) was added and the reaction mixture stirred at −78° C. for 2 hours. Carbon disulfide (0.16 mL) was added and the reaction mixture was stirred at −78° C. for 1 hour. The reaction mixture was warmed to room temperature. Merrifield's peptide resin (1.6 meq/g, 1.0 g) was added and the mixture stirred at room temperature overnight. The mixture was filtered. The resin was washed 5 times with 10 mL of acetone, 3 times with 10 mL of water, and twice with 10 mL of acetone. The resin was air dried (1.21 g).

The resin (1.21 g) and NaH (60% in oil, 0.003 g) were stirred in 20 mL of anhydrous DMF under argon for 4 hours. 1,3-Dibromopropane (0.07 mL) was added and the mixture stirred for 3 days. The mixture was filtered. The resin was washed 3 times with 10 mL of acetone, 5 times with 10 mL of water, and 5 times with 10 mL of acetone. The resin was air dried (1.22 g).

The resin (1.22 g) was resuspended and stirred in 20 mL of DMF under argon. Tributylphosphine (1.18 g) was added and the slurry stirred for 7 days. The slurry was filtered and the resin was washed 4 times with 20 mL of CH₂Cl₂ and 4 times with 20 mL of acetone. The resin was then air dried (1.07 g).

### Example 25

### General Procedure for Binding and Eluting DNA from Hydrolytically Cleavable Particles

A 10 mg sample of beads was rinsed with 500 μL of THF in a tube. The contents were centrifuged and the liquid removed. The rinse process was repeated with 200 μL of water. A solution of 2 μg of linearized pUC18 DNA in 200 μL of water was added to the beads and the mixture gently shaken for 20 min. The mixture was spun down and the supernatant collected. The beads were rinsed with 2×200 μL of water and the water discarded. DNA was eluted by incubating the beads with 200 μL of aq. NaOH at 37° C. for 5 min. The mixture was spun down and the eluent removed for analysis.

### Example 26

### Fluorescent Assay Protocol

Supernatants and eluents were analyzed for DNA content by a fluorescent assay using PicoGreen to stain DNA. Briefly, 10 μL aliquots of solutions containing or suspected to contain DNA are incubated with 190 μL of a fluorescent DNA “staining” solution. The fluorescent stain was PicoGreen (Molecular Probes) diluted 1:400 in 0.1 M tris, pH 7.5, 1 mM EDTA. Fluorescence was measured in a microplate fluorometer (Fluoroskan, Labsystems) after incubating samples for at least 5 min. The filter set was 480 nm and 535 nm. Positive controls containing a known amount of the same DNA and negative controls were run concurrently.

### Example 27

### Binding and Release of DNA from Cleavable Beads

Supernatants and eluents were analyzed for DNA content by a fluorescent assay using PicoGreen (Molecular Probes) to stain DNA. Results are expressed in comparison to the values obtained with an aliqout of the original 2 μg DNA solution. Analysis of wash solutions and supernatant from the binding step determined the % capture of DNA by the beads.

| Beads of Example # | [NaOH] (M) | % Bound | % Released |
|---|---|---|---|
| 11 | 0.005 | 36 | 33 |
| 13 | 1 | 100 | 100 |
| 14 | 1 | 36 | 100 |
| 15 | 1 | 100 | 100 |
| 18 | 1 | 100 | 78 |
| 19 | 0.1 | 100 | 100 |
| 20 | 0.05 | 100 | 79 |
| 21 | 1 | 100 | 77 |
| 22 | 1 | 100 | 72 |

### Example 28

### Effect of Elution Time and Temperature Toward Eluting DNA from Cleavable Particles

The beads of example 13 were treated according to the protocol of example 25. DNA-bound beads were incubated with 1 M NaOH at either room temperature or 37° C. for periods of 1, 5, or 10 minutes and the fraction of DNA released was determined by fluorescence.

| | | |
|---|---|---|
| Elution time | Room temp. | 37° C. |

| | | | |
|---|---|---|---|
| 1 | min | 80% | 100% |
| 5 | | 90 | 90 |
| 10 | | 90 | 120 |

### Example 29

### Binding and Release of DNA from Cleavable Beads Using a Spin Column

A solution of 2 μg of linearized pUC18 DNA in 200 μL of water was added to 20 mg of beads in a 2 mL spin column (Costar). After incubation for 2 min the column was spun down for 30 s and the supernatant collected. The beads were washed with 2×200 μL of water and the washes discarded. DNA was eluted by washing the beads with 200 μL of 0.5 M NaOH at 37° C. for 1 min, spinning for 30 s and collecting the eluent for analysis by fluorescence and gel electrophoresis. DNA eluted was amplified by PCR using the eluent directly without precipitating the DNA.

### Example 30

### PCR Amplification of Plasmid DNA Bound and Released from Cleavable Beads of Example 13

The eluted DNA of the previous example (1 μL) in 0.5 M NaOH was subject to PCR amplification with a pair of primers which produced a 285 bp amplicon. PCR reaction mixtures contained the components listed in the table below.

| Component | Volume (μL) |
|---|---|
| 10× PCR buffer | 10 |
| Primer 1 | 8 |
| Primer 2 | 8 |
| 2.5 mM dNTPs | 8 |
| 50 mM MgCl₂ | 5 |
| Taq DNA polymerase | 0.5 |
| Template | 1 or 2 |
| deionized water | 59.5 or 58.5 |

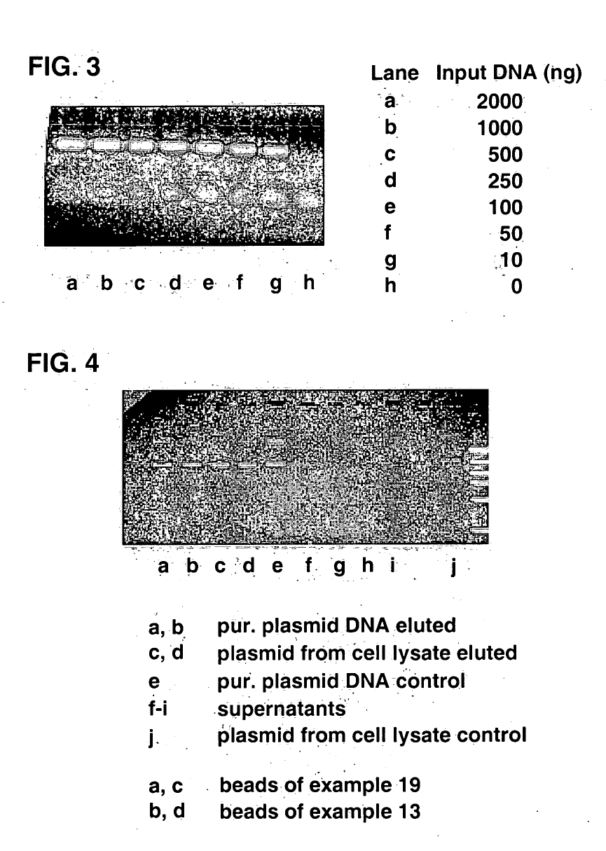

Negative controls replaced template in the reaction mix with 1 or 2 μL of 0.5 M NaOH or 1 μL of water. A further reaction used 1 μL of template diluted 1:10 in water. Reaction mixtures were subject to 22 cycles of 94° C., 1 min; 60° C., 1 min; 72° C., 1 min. Reaction products were run on 1% agarose gel. FIG. 3 demonstrates that the DNA eluted from the beads is intact.

### Example 31

### Binding of Oligonucleotides of Different Lengths with Tributylphosphonium Beads of Example 13 and Release with 1 M NaOH

The binding and release protocol of example 25 was performed on various size oligonucleotides ranging from 20 bases to 2.7 kb. The beads were cleaved with 200 μL of 1 M NaOH at 37° C. for 5 min. The amount of DNA was determined fluorometrically using OliGreen, a fluorescent stain for ssDNA.

| | |
|---|---|
| Oligonucleotide size (nt) | % Eluted |

| | | |
|---|---|---|
| 20 | | 61 |
| 30 | | 65 |
| 50 | | 64 |
| 68 | | 48 |
| 181 | | 47 |
| 424 | | 52 |
| 753 | | 70 |
| 2.7 | kb | 51 |

A repeat of the experiment using a 30 min reaction of beads at room temperature to cleave the polymer produced the results below.

| Oligonucleotide size (nt) | % Eluted |
|---|---|
| 20 | 73 |
| 30 | 113 |
| 50 | 97 |
| 68 | 109 |

### Example 32

### Binding and Release of DNA from Magnetic Cleavable Beads of Example 16

A solution of 2 μg of linearized pUC18 DNA in 200 μL of water was added to 10 mg of the cleavable magnetic beads and the mixture gently shaken for 20 min. The mixture was separated magnetically and the supernatant collected. The beads were rinsed with 2×200 μL of water and the water discarded. DNA was eluted by incubating the beads with 2×200 μL of 0.5 M NaOH at 37° C. for 5 min. The mixture was spun down and the eluent removed for fluorescence analysis. All of the DNA was bound to the beads. The first eluent contained 92% of the bound DNA; the second contained 13%.

### Example 33

### Binding and Release of DNA from Magnetic Cleavable Beads of Example 17

Following the same procedure, the cleavable magnetic beads of example 17 were used to bind and release 2 μg of linearized pUC18 DNA. Analysis of supernatants from the binding step revealed that the DNA was completely bound. Analysis of the eluents after release from the beads showed the intact DNA to be eluted.

### Example 34

### Binding Capacity of Magnetic Beads of Example 16

Various quantities of DNA listed in the table below were bound to the cleavable magnetic beads of example 16 and eluted as described above with 0.5 M NaOH. Supernatants and eluents were assayed fluorometrically to assess the binding capacity and ability to release different amounts of DNA.

| Amount of input DNA | % bound | % eluted |
|---|---|---|
| 2 | 100 | 83 |
| 4 | 100 | 83 |
| 6 | 100 | 84 |
| 10 | 100 | 90 |
| 14 | 100 | 100 |

### Example 35

### Releasing DNA Bound on Cleavable Beads of Example 13 with Smaller Elution Volume

A solution of 2 μg of linearized pUC18 DNA in 200 μL of water was added to 10 mg of beads in a 2 mL spin column (Costar). After incubation for 5 min the column was spun down for 1 min and the supernatant collected. The beads were washed with 2×200 μL of water and the washes discarded. DNA was eluted three times by washing the beads each time with 40 μL of 0.5 M NaOH at 37° C. for 5 min, spinning for 30 s and collecting the eluent for analysis by fluorescence and gel electrophoresis after each elution. All of the starting DNA was bound. The elutions were found to contain 65%, 22%, and 9% respectively.

### Example 36

### Binding DNA from Large Volumes onto Cleavable Magnetic Beads of Example 16 and Releasing with Small Elution Volume

A solution of 2 μg of linearized pUC18 DNA in either 1 mL, 2 mL or 10 mL of water was added to 10 mg of the cleavable magnetic beads of example 16 and eluted as described above with 200 μL of 0.5 M NaOH at 37° C. for 5 min. Supernatants from the 1 mL and 2 mL binding reactions were concentrated to ca. 100 μL for analysis. Eluents from all three runs were assayed fluorometrically as well. The supernatants contained no DNA. All eluents contained >80% of the starting DNA.

### Example 37

### Isolation of DNA from Bacterial Culture with Polymer Beads of Example 13

An E. coli culture was grown overnight. A 50 mL portion was centrifuged at 6000×g for 15 min at 4° C. to pellet the cells. The pellet was resuspended in 4 mL of 50 mM tris, pH 8.0, 10 mM EDTA, containing 100 μg/mL RNase A. Then 4 mL of 0.2 M NaOH solution containing 1% SDS was added to the mixture which was gently mixed and kept for 4 min at room temperature. Next, 4 mL of 3 M KOAc, pH 5.5, cooled to 4° C., was added, the solution mixed and allowed to stand for 10 min to precipitate SDS. The precipitate was filtered off and the filtrate was collected.

Lysate diluted 1:10 in water (200 μL) was mixed with 10 mg of the beads of example 13 and incubated for 20 min. A solution of purified pUC18, 0.33 g/200 μL in cell lysate medium, was also prepared and bound to 10 mg of the same beads. After binding the beads were spun down and the supernatants removed. The bead samples were washed with 2×200 μL of water and then eluted with 200 μL of 5 mM NaOH at 37° C. for 5 min. Gel electrophoresis shows recovery of plasmid DNA from lysate-which matches plasmid controls either bound to beads and released or in free solution. Results are shown in FIG. 4.

### Example 38

### Isolation of DNA from Bacterial Culture with Polymer Beads of Example 19

DNA in the cell lysate of the previous example was isolated using the beads of example 19 according to the same protocol described above in example 37. Results are shown in FIG. 4.

### Example 39

### Binding DNA onto Beads of Example 13 from Different pH Solutions Showing Effective Capture Over a Wide Range of pH

Buffers spanning the pH range 4.5 to 9.0 were prepared. Buffers having pH 4.5 to 6.5 were 10 mM acetate buffers. Buffers having pH 7.0 to 9.0 were 10 mM tris acetate buffers. A solution of 2 μg of linearized pUC18 DNA in 200 μL of each buffer was added to 10 mg of the cleavable beads of example 13 for 30-45 s at room temperature. Negative control solutions with no DNA in each buffer were run in parallel. Supernatants were removed after spinning bead samples down and analyzed by UV and fluorescence.

| Buffer pH | % Bound (by UV) | % Bound (by Fl.) |
|---|---|---|
| 4.5 | 56 | 73 |
| 5.0 | 64 | 68 |
| 5.5 | 58 | 64 |
| 6.0 | 61 | 71 |
| 6.5 | 57 | 74 |
| 7.0 | 49 | 61 |
| 7.5 | 44 | 60 |
| 8.0 | 45 | 55 |
| 8.5 | 37 | 39 |
| 9.0 | 31 | 33 |

Separately it was found that binding for 5 min using 20 mg of beads at pH 8.0 resulted in 100% capture of DNA.

### Example 40

### Release of DNA from cleavable beads by use of different basic solutions for hydrolysis

A solution of 2 μg of linearized pUC18 DNA in 200 μL of water was added to 10 mg of the cleavable beads of example 13, 18, 19 and 20 and eluted with 200 μL of NaOH solutions of various concentrations listed below at 37° C. for 5 min. The beads of example 13 were also cleaved with KOH and NH₄OH solutions. Eluents from all runs were assayed by gel. All hydrolysis conditions tested resulted in cleavage and release of DNA.

| Base | Concentration (M) |
|---|---|
| NaOH | 0.005 |
| ″ | 0.01 |
| ″ | 0.05 |
| ″ | 0.1 |
| ″ | 0.5 |
| ″ | 1.0 |
| KOH | 0.5 |
| NH₄OH | 0.5 |
| ″ | 1.0 |

### Example 41

### Binding and Release of DNA from Cleavable Beads of Example 8-Br, and 8-S

A 25 mg sample of each of the two kinds of beads was rinsed with 500 μL of THF in a tube. The contents were centrifuged and the liquid removed. The rinse process was repeated with 500 μL of water. A solution of 16 μg of linearized pUC18 DNA in 500 μL of water was added to the beads and the mixture gently shaken for 20 min. The mixture was spun down and the supernatant collected. The beads were rinsed with 2×500 μL of water and the water discarded. DNA was eluted by incubating the beads with 500 μL of 1 M NaOH at 37° C. for 16 h. The mixture was spun down and the eluent removed for analysis by fluorescence. The supernatants contained no DNA, all was bound. The eluents were found to contain 18% (8-Br) and 12% (8-S).

### Example 42

### Use of DNA Eluted from Cleavable Beads of Example 13 in LMO Amplification

Solutions containing 0.1 or 1 μg of pUC18 DNA in 200 μL of water were added to 10 mg of beads previously washed with 400 μL of THF and then twice with water. After incubation for 30 min the sample tubes were spun down for 30 s and the supernatants collected. The beads were washed with 2×400 μL of water and the washes discarded. DNA was eluted by washing the beads with 100 μL of 1 M NaOH at room-temperature for 15 min, spinning for 30 s and collecting the eluent. An 80 μL portion of each eluent was neutralized with 40 μL of 1 M acetic acid.

Plasmid DNA isolated using the polymeric beads of the invention was amplified by LMO as described in U.S. Pat. No. 5,998,175 using the eluent directly without precipitating the DNA. Briefly, a 68 bp region was amplified by a thermocycling protocol using a pair of primers and a set of octamers spanning the 68 base region. A set of twelve octamer-5′-phosphates (six per strand), the primers and template (1 μL) were dissolved in Ampligase buffer. Reaction tubes were overlaid with 50 μL of mineral oil and heated to 94° C. for 5 min. After about 2 min 100 U of Ampligase was added to each tube. Samples were cycled 35 times at 94° C. for 30 s; 55° C. for 30 s; 35° C. for 30 s. Gel electrophoresis of the amplification reactions revealed a band of the expected molecular weight.

### Example 43

### Isolation of human genomic DNA from whole blood using cleavable beads of example 13

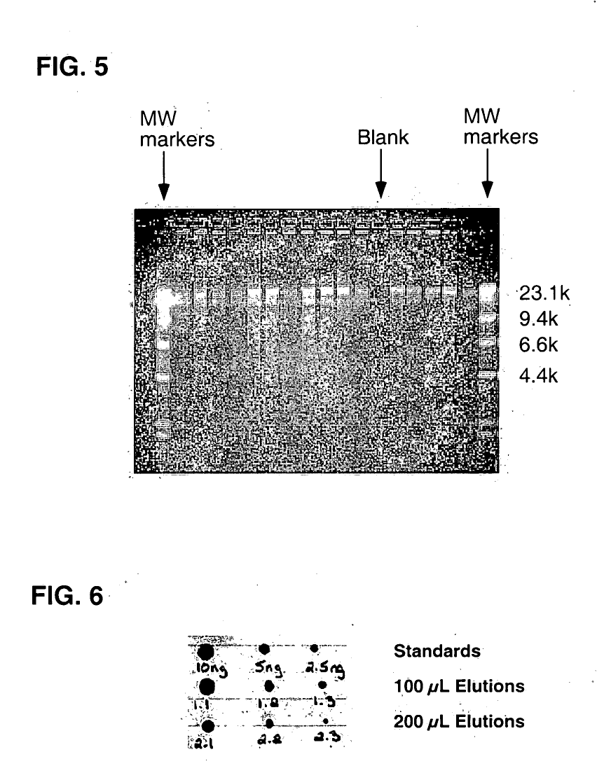

Pelleted white blood cells from 16 human blood samples (1-3 mL) prepared by standard protocols were suspended in 100 μL of a lysis buffer comprising 0.2 M tris, pH 8.0, 0.1 M EDTA, 1% SDS. Proteinase K (10 μg) was added to each tube and the tubes incubated at 55° C. for 4 h. 3M KOAc (100 μL) was added to each tube and the tubes mixed by gentle inversion. The tubes were spun down at 13,000 rpm. Supernatant was removed and diluted 1:2 with water. DNA in the solutions was bound to 10 mg of beads for 20 min at room temperature. After binding the beads were spun down and the supernatants removed. The bead samples were washed with 2×200 μL of water and then eluted with 200 μL of 5 mM NaOH at 37° C. for 5 min. Samples of each eluent were analyzed by agarose gel electrophoresis. FIG. 5 show the recovery of high molecular weight DNA from all samples.

### Example 44

### Binding and Release of DNA on Acridan Ketene Dithioacetal Polymer of Example 24 by Enzymatic Reaction

A 60 mg sample of beads was rinsed with 500 μL of THF in a tube. The contents were centrifuged and the liquid removed. The rinse process was repeated with 400 μL of water. A solution of 2 μg of linearized pUC18 DNA in 250 μL of water was added to the beads and the mixture gently shaken for 20 min. The mixture was spun down and the supernatant collected. The beads were rinsed with 2×200 μL of water and the water discarded.

DNA was eluted by enzymaticaly oxidizing the acridan linker moiety with HRP and peroxide. A composition containing 14 fmol of HRP in 0.025 M tris, pH 8.0, 4 mM p-hydroxycinnamic acid, 2.5 mM urea peroxide, 0.1% Tween-20, 0.5 mM EDTA. A control composition lacking the HRP was run-in parallel. The reactions of the beads with the compositions were run for 1 h at room temperature. Solutions were analyzed for DNA content by fluorescence assay and by gel electrophoresis. Analysis of supernatants showed 100% binding of DNA. Analysis of eluents showed 52% of bound DNA was eluted in the enzymatic reaction; no DNA was eluted in the control.

### Example 45

### Binding and Release of DNA on Acridinium Ester Polymer of Example 23

A 100 mg sample of beads was rinsed with 1 mL of THF in a tube. The contents were centrifuged and the liquid removed. The rinse process was repeated with 2×1 mL of water. A solution of 75 μg of pUC18 DNA in 586 μL of water was added to the beads and the mixture gently shaken for 2 h at room temperature. A negative control sample of beads containing no DNA was processed in parallel. The mixture was spun down and the supernatant collected. The beads were rinsed with 2×1 mL of water and the water discarded. UV analysis of supernatants showed that the beads had bound 10% of the DNA. DNA was eluted by reaction with 200 μL of 1 M NaOH containing 1 M urea peroxide for 30 min at room temperature. Beads were separated from the eluent and the eluents neutralized with 1 M acetic acid. Analysis of the neutralized eluents by dot blot showed a small amount of DNA to be released. The negative control showed no signal.

### Example 46

### Binding of DNA to Polymer Beads of Example 9

A 100 mg sample of beads was rinsed with 1 mL of THF in a tube. The contents were centrifuged and the liquid removed. The rinse process was repeated twice with 1 mL of water. A solution of 80 μg of pUC18 DNA in 1 mL of water was added to the beads and the mixture gently shaken for 20 min. The mixture was spun down and the supernatant collected for UV analysis. The supernatant contained 66 μg of DNA. The binding capacity was thus determined to be 0.14 μg/mg.

### Example 47

### Binding and Release of RNA from Cleavable Beads of Example 13

In two tubes, 2 μg of Luciferase RNA was bound to 10 mg of beads. 1×Reverse transcriptase buffer (50 mM tris-HCl, pH 8.5, 8 mM MgCl₂, 30 mM KCl, 1 mM DTT) was used for elution. One tube was heated for 5 min at 94° C. and the other-tube was heated for 30 min at 94° C. The eluents and controls were run on a 1% agarose gel and stained with SYBR Green™. The 5 min heating showed ˜50% elution of RNA from the beads but the 30 min heating seemed to denature the RNA.

### Example 48

### Binding and Release of RNA from Cleavable Beads of Example 13 with Different Cleavage/Elution Buffers

In three tubes, 1 μg of Luciferase RNA was bound to 10 mg of beads. In one tube, 3M potassium acetate was used to elute the RNA at room temperature for 30 min. In another tube, 1× reverse transcriptase buffer (RT) was used for elution at 94° C. for 1 min. The third tube had RNA extraction buffer and was heated to 94° C. for 1 min. RNA extraction buffer consists of 10 mM tris-HCl, pH 8.8, 0.14 M NaCl, 1.5 M MgCl₂, 0.5% NP-40, 1 mM DTT. All eluents and controls were run on a 1% agarose gel and stained with SYBR Green™. The 3M potassium acetate did not produce recognizable RNA. The 1× reverse transcriptase buffer and RNA extraction buffer both showed a band estimated to contain RNA corresponding to about 50% elution.

### Example 49

### Binding and Release of Rna from Cleavable Beads of Example 13 and Detection by Chemiluminescent Blot Assay

In four tubes, 1 μg of Luciferase RNA was bound to 10 mg of beads. Two tubes used the 1× reverse transcriptase buffer for elution and the other two used RNA extraction buffer. One tube of each kind of buffer was heated to 94° C. for 1 min. The other two tubes were heated to 94° C. for 5 min. All eluents and controls were run on a 1% agarose gel and stained with SYBR Green. The eluents heated 1 min contained more RNA than those heated for 5 min using either buffer. RNA extraction buffer eluted more RNA than the 1×RT buffer. The RNA was transferred onto a nylon membrane with an overnight capillary transfer. The RNA was then hybridized overnight with HF-1 biotin labeled primer. Detection was done with anti-biotin HRP and Lumigen PS-3 as chemiluminescent substrate. The 5 min exposure verified the gel results.

### Example 50

### Binding and Release of RNA from Cleavable Beads of Example 13 at Various Temperatures

In six tubes, 1 μg of Luciferase RNA was bound to 10 mg of beads. RNA extraction buffer was used to elute the RNA for 5 min at several different temperatures: 40° C., 50° C., 60° C., 70° C., 80° C., and 90° C. All eluents and controls were run on a 1% agarose gel and stained with SYBR Green. All temperatures appeared to elute 100%.

### Example 51

### Binding of Linearized Puc18 DNA with Tributyl-Phosphonium Beads of Example 1 and Release with Different Elution Compositions

A 10 mg sample of beads was rinsed with 500 μL of THF in a tube. The contents were centrifuged and the liquid removed. The rinse process was repeated with 200 μL of water. A solution of 2 μg of linearized pUC18 DNA in 200 μL of water was added to the beads and the mixture gently shaken for 20 min. The mixture was spun down and the supernatant collected. The beads were rinsed with 2×200 μL of water and the water discarded. DNA was eluted by incubating the beads with 200 μL of various reagent compositions described in the table below at room temperature for 20 min. The mixture was spun down and the eluent removed for fluorescence analysis as described in example 26.

| Buffer | Salt | Org. Solvent | % Eluted |
|---|---|---|---|
| 50 mM tris, pH 8.5 | 1.25 M NaCl | 15% furfuryl | 58 |
| | | alcohol | |
| 50 mM tris, pH 8.5 | 1.25 M NaCl | 15% ficoll | 19 |
| 50 mM tris, pH 8.5 | 1.25 M NaCl | 15% HOCH₂CH₂SH | 52 |
| 50 mM tris, pH 8.5 | 1.25 M NaCl | 15% DTT | 52 |
| 50 mM tris, pH 8.5 | 1.25 M NaCl | 15% glycerol | 15 |
| 50 mM tris, pH 8.5 | 1.25 M NaCl | 15% 2-propanol | 50 |
| 50 mM tris, pH 8.5 | 1.25 M NaCl | 15% ethanol | 37 |
| 50 mM tris, pH 8.5 | 1.25 M NaCl | 15% CF₃CH₂OH | 38 |
| 50 mM tris, pH 8.5 | 1.25 M NaCl | 15% acetone | 42 |
| 50 mM tris, pH 8.5 | 1.25 M NaCl | 15% THF | 41 |
| 50 mM tris, pH 8.5 | 1.25 M NaCl | 15% p-dioxane | 33 |

### Example 52

The bind and release protocol of example 51 was followed with reagent compositions described in the table below. The effect of changing the concentration of either DTT or 2-mercaptoethanol was examined.

| Buffer | Salt | Org. Solvent | % Eluted |
|---|---|---|---|
| 50 mM tris, pH 8.5 | 1.25 M NaCl | 0.1% DTT | 0 |
| 50 mM tris, pH 8.5 | 1.25 M NaCl | 1% DTT | 0 |
| 50 mM tris, pH 8.5 | 1.25 M NaCl | 3% DTT | 36 |
| 50 mM tris, pH 8.5 | 1.25 M NaCl | 4% DTT | 41 |
| 50 mM tris, pH 8.5 | 1.25 M NaCl | 0.1% HOCH₂CH₂SH | 0 |
| 50 mM tris, pH 8.5 | 1.25 M NaCl | 1% HOCH₂CH₂SH | 0 |
| 50 mM tris, pH 8.5 | 1.25 M NaCl | 3% HOCH₂CH₂SH | 39 |
| 50 mM tris, pH 8.5 | 1.25 M NaCl | 4% HOCH₂CH₂SH | 38 |

### Example 53

The bind and release protocol of example 51 was followed with reagent compositions described in the table below. The effect of changing the concentration of salts NaCl and KCl was examined.

| Buffer | Salt | Org. Solvent | % Eluted |
|---|---|---|---|
| 50 mM tris, pH 8.5 | 0.1M NaCl | 5% DTT | 1 |
| 50 mM tris, pH 8.5 | 0.25 M NaCl | 5% DTT | 0 |
| 50 mM tris, pH 8.5 | 0.5 M NaCl | 5% DTT | 27 |
| 50 mM tris, pH 8.5 | 0.75 M NaCl | 5% DTT | 29 |
| 50 mM tris, pH 8.5 | 1.0 M NaCl | 5% DTT | 29 |
| 50 mM tris, pH 8.5 | 1.25 M NaCl | 5% DTT | 26 |
| 50 mM tris, pH 8.5 | 0.75 M KCl | 5% DTT | 64 |
| 50 mM tris, pH 8.5 | 1.25 M KCl | 5% DTT | 60 |

### Example 54

The bind and release protocol of example 51 was followed with reagent compositions described in the table below. Beads were eluted for 60 min.

| Buffer | Salt | Org. Solvent | | % Eluted |
|---|---|---|---|---|
| 50 mM tris, pH 8.5 | 0.1 M NaCl | 0% | 2-propanol | 3 |
| 50 mM tris, pH 8.5 | 0.1 M NaCl | 15% | 2-propanol | 68 |
| 50 mM tris, pH 8.5 | 0.25 M NaCl | 30% | 2-propanol | 64 |
| 50 mM tris, pH 8.5 | 0.5 M NaCl | 50% | 2-propanol | 4 |

### Example 55

The bind and release protocol of example 51 was followed with reagent compositions described in the table below. Relative effectiveness is scored.

| Buffer | Salt | Org. Solvent | |
|---|---|---|---|
| 50 mM tris, pH 8.5 | 1.0 M Na acetate | 15% 2-propanol | ++ |
| 50 mM tris, pH 8.5 | 1.5 M Na acetate | 15% 2-propanol | ++ |
| 50 mM tris, pH 8.5 | 1.25 M Na acetate | 15% 2-propanol | ++ |
| 50 mM tris, pH 8.5 | 0.75 M Na acetate | 15% 2-propanol | + |
| 50 mM tris, pH 8.5 | 0.5 M Na acetate | 15% 2-propanol | + |
| 50 mM tris, pH 8.5 | 0.1 M Na acetate | 15% 2-propanol | + |

### Example 56

### Binding of oligonucleotides of different lengths with tributylphosphonium beads of example 1 and release with a reagent composition

The bind and release protocol of example 51 was performed on various size oligonucleotides ranging from 20 bases to 2.7 kb. The elution composition was 50 mM tris, pH 8.5, 0.75 M NaCl, 5% DTT. The amount of DNA was determined fluorometrically using OliGreen, a fluorescent stain for ssDNA.

| Oligonucleotide size (nt) | | % Eluted |
|---|---|---|
| 20 | | 39 |
| 30 | | 43 |
| 50 | | 36 |
| 68 | | 34 |
| 181 | | 33 |
| 424 | | 33 |
| 753 | | 32 |
| 2.7 | kb | 20 |

### Example 57

### Binding of Linearized PUC18 DNA with Tributyl-Phosphonium Beads of Example 1 and Release with Different Elution Volumes

A solution of 2 μg of linearized pUC18 DNA in 200 μL of water was added to 10 mg of beads in a 2 mL spin column (Costar). After incubation for 20 min the column was spun down and the supernatant collected. The beads were washed with 2×200 μL of water and the washes discarded. DNA was eluted by washing the beads with 5×200 μL of 50 mM tris, pH 8.5, 0.75 M NaCl, 5% DTT at room temperature for 5 min, spinning and collecting the eluent for analysis by fluorescence and gel electrophoresis after each elution.

In a similar manner, beads containing bound DNA were eluted with 5×40 μL of the same elution buffer.

| | 200 μL elutions | 40 μL elutions |
|---|---|---|
| Elution 1 | 63 | 47 |
| Elution 2 | 10 | 11 |
| Elution 3 | 5.5 | 10 |
| Elution 4 | 3.5 | 5 |
| Elution 5 | 2.1 | 4 |
| Total | 84 | 77 |

### Example 58

### Binding and Release of Nucleic Acid with Tributylammonium Beads of Example 5

A solution of 2 μg of linearized pUC18 DNA in 200 μL of water was added to 10 mg of beads and the mixture gently shaken for 30 min. The mixture was spun down and the supernatant collected. The beads were rinsed with 2×200 μL of water and the water discarded. DNA was eluted by incubating the beads with 200 μL of 50 mM tris, pH 8.5, 0.75 M NaCl, 5% DTT at room temperature for 30 min. The mixture was spun down and the eluent removed for fluorescence analysis as described in example 26. DNA binding was 50%, elution was 69% of the bound portion.

### Example 59

### Binding and Release of Nucleic Acid with Magnetic Tributylphosphonium Beads of Example 7

A 10 mg sample of beads was rinsed with 500 μL of THF in a tube. The contents were magnetically separated and the liquid removed. The rinse process was repeated with 200 μL of water. A solution of 2 μg of linearized pUC18 DNA in 200 μL of water was added to the beads and the mixture gently shaken for 20 min. The mixture was separated magnetically and the supernatant collected. The beads were rinsed with 2×200 μL of water and the water discarded. DNA was eluted by incubating the beads with 200 μL of 50 mM tris, pH 8.5, 1.25 M NaCl, 15% 2-propanol at room temperature for 30 min. The mixture was separated magnetically and the eluent removed for fluorescence analysis as described in example 26. DNA binding was 100%, elution was 18%.

### Example 60

### Binding of Linearized PUC18 DNA with Tributyl-Phosphonium Beads of Example 1 and Release with Different Elution Temperatures

A solution of 2 μg of linearized pUC18 DNA in 200 μL of water was added to 10 mg of beads and the mixture gently shaken for 30 min. The mixture was spun down and the supernatant collected. The beads were rinsed with 2×200 μL of water and the water discarded. DNA was eluted by incubating the beads with 200 μL of 50 mM tris, pH 8.5, 1.25 M NaCl, 15% 2-propanol for 5 min at various temperatures: 37° C., 46° C., 65° C., and 94° C. The mixture was spun-down and the eluent removed for fluorescence analysis as described in example 26. DNA binding was 100%, elution was ca. 65-70% of the bound portion at all temperatures.

### Example 61

### PCR Amplification of Plasmid DNA Bound and Released from Beads of Example 1

Following the protocol of example 51, 1 μL of the eluted plasmid DNA in 0.5 M NaOH was subject to PCR amplification with a pair of primers spanning a 285-base region. PCR reaction mixtures contained the components listed in the table below.

| Component | Volume (μL) |
|---|---|
| 10× PCR buffer | 10 |
| Primer 1 (1.5 pmol/μL) | 8 |
| Primer 2 (1.5 pmol/μL) | 8 |
| 2.5 mM dNTPs | 8 |
| 50 mM MgCl₂ | 5 |
| Taq DNA polymerase | 0.5 |
| Template | 1 or 2 |
| deionized water | 59.5 or 58.5 |

Negative controls replaced template in the reaction mix with 1 or 2 μL of 0.5 M NaOH or 1 μL of water. A further reaction used 1 μL of template diluted 1:10 in water. Reaction mixtures were subject to 22 cycles of 94° C., 1 min; 60° C., 1 min; 72° C., 1 min. Reaction products were run on 1% agarose gel which demonstrated that the DNA eluted from the beads was intact.

### Example 62

### Binding of Nucleic Acids with Tributylphophonium Beads of Example 1 and Release by a Wittig Reaction

A solution of 2 μg of pUC18 DNA in 200 μL of water was added to 10 mg of the beads of example 1 and the mixture gently shaken for 20 min. The mixture was spun down and the supernatant collected. The beads were rinsed with 2×200 μL of water and the water discarded. The beads were washed with 5×400 μL of DMF. A saturated solution of NaOt—Bu in DMF (300 μL) and 20 μL of acetone were shaken with the beads for 20 min. The mixture was spun down and the liquid removed. The beads were washed with 3×400 μL of DMF, the liquid removed after the last wash. DNA was eluted by shaking the beads with 200 μL of 10 mM tris, pH 8.5 for 5 min and collecting the solution. The process was repeated twice with fresh portions of buffer.

### Example 63

### Dot Blot Analysis of Wittig Released DNA

Portions (1 μL) of the three elutions of example 62 after Wittig reaction were analyzed by dot blot on nylon membrane. DNA applied to the membrane was UV crosslinked and rinsed with 2×SSC buffer. The membrane was prehybridized with 5 mL of Dig Easy Hyb™ buffer (Roche) for 1.5 h at 37° C. Digoxigenin labeled 30mer probe was hybridized overnight in Dig Easy Hyb buffer at 37° C. Hybridized probe was captured with anti-digoxigenin HRP conjugate (1:10,000 dilution) in 2% BM block solution (Boehringer-Mannheim) for 1 h. HRP label was detected by wetting the membrane with Lumigen PS-3 and exposing to x-ray film. Standards containing 10, 5 and 2.5 ng of DNA were analyzed in parallel with the eluted samples and supernatants from the binding step. FIG. 6 demonstrates that the most bound DNA was removed in the first elution, with progressively smaller amounts removed in the second and third elutions. Analysis of the supernatants (not shown) demonstrated that all of the DNA was bound to the beads. Similar experiments in which released DNA was eluted at 100° C. gave similar results.

### Example 64

Effect of Reaction Time on Removal of Released DNA in Protocol of Example 62

The protocol of example 62 was performed with modification of the reaction time in the Wittig reaction with acetone. In separate experiments reaction times of 10 min, 20 min, 30 min and 60 min were used. Dot blot analysis as described in example W2 demonstrated that equivalent results were obtained regardless of reaction time.

### Example 65

### Binding of Nucleic Acids with Trimethyl-Phosphonium Beads of Example 3 and Release by a Wittig Reaction

The beads of example 3 were used to bind DNA and released by Wittig according to the general method described in example 62. Analysis by UV of supernatants from the binding step showed that 78% of DNA was captured. The binding capacity is 0.156 μg/mg, compared to >0.2 μg/mg for the tributylphosphonium beads. Similar to the tributylphosphonium beads, the most DNA was removed from the beads in the first-elution.

### Example 66

### Binding of Nucleic Acids with Triphenyl-Phosphonium Beads of Example 4 and Release by a Wittig Reaction

The beads of example 4 were used to bind 17 μg of DNA on 25 mg of beads and to release by Wittig reaction according to the general method described in example 62. Analysis by UV of supernatants from the binding step showed that 14% of DNA was captured. The binding capacity is 0.095 μg/mg. Similar to the tributylphosphonium beads, the most DNA was removed from the beads in the first elution.

### Example 67

### Binding of Nucleic Acids with Magnetic Tributylphosphonium Beads of Example 7 and Release by a Wittig Reaction

The protocol of example 62 was followed with the following modifications. All separation steps were performed-magnetically. Organic solvent and washes substituted THF in place of DMF. The volume of THF/NaOt—Bu solution was 250 μL. Released DNA was eluted with three 15 min washes in tris buffer. Eluents and supernatants were analyzed by fluorescent assay with PicoGreen. Analysis of supernatants showed 100% binding to particles. Fluorescent assay found 32% eluted in the first elution. Subsequent elutions contained too little DNA to detect by this method. For comparison, the nonmagnetic beads of example 1 showed 31% DNA in the first elution and too little to detect in subsequent elutions.

### Example 68

### Use of DNA Eluted from Cleavable Beads of Example 16 Directly in LMO and PCR Amplification

Solutions containing 4 μg of genomic DNA isolated from whole human blood in 200 μL of 10 mM tris, pH 8.5 were added to 20 mg of beads. After incubation for 5 min the sample tubes were spun down for 30 s and the supernatants collected. The beads were washed with 2×200 μL of water and the washes discarded. DNA was eluted by washing the beads with 100 μL of 0.5 M NH₄OH at 37° C. for 5 min, spinning for 30 s and collecting the eluent.

DNA isolated using the polymeric beads of the invention was amplified without neutralization or further sample pretreatment by LMO as described in U.S. Pat. No. 5,998,175. Briefly, an amplicon corresponding to a segment of the Factor V gene was prepared which had a 51 base strand and a 48 base complement by a thermocycling protocol using a pair of primers, one of which was 5′-labeled with 6-FAM, and a set of two octamers and two decamers. The primers and template (1 μL) were dissolved in Taq DNA ligase buffer. Reaction tubes were overlaid with 40 μL of mineral oil and heated to 94° C. for 5 min. Then 20 U of Taq DNA ligase was added to each tube. Samples were cycled 40 times at 94° C. for 30 s; 55° C. for 30 s; 38° C. for 30 s.

A chemiluminescent hybridization assay of the amplification reactions was performed. A Capture probe for the wild type amplicon was immobilized in microplate wells and used to hybridize to amplification product containing the FAM label. Anti FITC-alkaline phosphatase conjugate was bound and detected with Lumi-Phos Plus. DNA from blood samples of each genotype and a water blank were run in parallel through the LMO, hybridization and detection steps. The amount of DNA in the known controls was chosen to equal the amount in the bead processed samples at 50% recovery. The sample had been previously typed as homozygous wt.

| Specimen | Signal (RLU) |
|---|---|
| Sample | 24.7 |
| Homozygous wt | 87.3 |
| Heterozygous | 47.1 |
| Homozygous mut | 0.20 |
| Blank | 0.30 |

### Example 69

### Synthesis of Polymethacrylate Polymer Containing Dimethylsulfonium Groups and Arylthioester Linkage

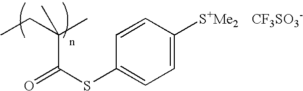

Polymethacryloyl chloride resin, prepared as described above, (2.96 g), 5.07 g of 4-(methylthio)thiophenol and triethylamine (8.8 mL) were stirred in 100 mL of CH₂Cl₂ at room temperature under argon for 5 days. The solid was filtered off and washed with 100 mL of CH₂Cl₂ and 100 mL of water and then was stirred in 125 mL of methanol for several days. Filtration and drying yielded 3.76 g of the thioester product.

A 2.89 g portion of the solid in 100 mL of CH₂Cl₂ was stirred with 4.1 mL of methyl triflate for 7 days. The solid was filtered and washed sequentially with 200 mL of CH₂Cl₂, 300 mL of methanol and 300 mL of CH₂Cl₂ and then air dried.

### Example 70

### Binding and Release of DNA Using Cleavable Beads Having Dimethylsulfonium Group

A solution of 2 μg of linearized pUC18 DNA in 200 μL of 10 mM tris, pH 8 was added to a 10 mg sample of the beads of example 69 and the mixture gently shaken for 5 min. The mixture was spun down and the supernatant collected. The beads were rinsed with 2×200 μL of water and the water discarded. DNA was eluted by incubating the beads with 200 μL of 0.5 M. NaOH at 37° C. for 5 min. The mixture was spun down and the eluent removed for fluorescence analysis. The supernatant contained no DNA. The eluent contained 100% of the initially bound DNA.

### Example 71

### Binding and Release of DNA Using Cleavable Beads Having Dimethylsulfonium Group

DNA bound to beads as described in example 70 was eluted by incubating with 200 μL of 50 mM tris, pH 8.5, 0.75 M NaCl, 5% DTT at 37° C. for 5 min. The mixture was spun down and the eluent removed for fluorescence analysis. The supernatant contained no DNA. The eluent contained 37% of the initially bound DNA.

The foregoing description and examples are illustrative only and not to be considered restrictive. It is recognized that modifications of the specific compounds and methods not specifically disclosed can be made without departing from the spirit and scope of the present invention. The scope of the invention is limited only by the appended claims.

1. A method of isolating a nucleic acid from a sample comprising:
a) providing a solid phase comprising:
a solid support portion comprising a matrix selected from silica, glass, insoluble synthetic polymers, and insoluble polysaccharides,
a nucleic acid binding portion for attracting and binding nucleic acids, and
a cleavable linker portion;
b) combining the solid phase with the sample containing the nucleic acid to bind the nucleic acid to the solid phase;
c) separating the sample from the solid phase;
d) cleaving the cleavable linker; and
e) releasing the nucleic acid from the solid phase.

2. The method of claim 1 wherein the nucleic acid binding portion of the solid phase is selected from a ternary sulfonium group of the formula SR₂⁺X⁻ where R is selected from C₁-C₂₀ alkyl, aralkyl and aryl groups, a quaternary ammonium group of the formula NR₃⁺X⁻ wherein R is selected from C₄-C₂₀ alkyl, aralkyl and aryl groups, and a quaternary phosphonium group PR₃⁺X⁻ wherein R is selected from C₁-C₂₀ alkyl, aralkyl and aryl groups, and wherein X is an anion.

3. The method of claim 2 wherein the nucleic acid binding portion is a quaternary ammonium group and the R groups each contain from 4-20 carbon atoms.

4. The method of claim 2 wherein the nucleic acid binding portion is a quaternary phosphonium group and the R groups each contain from 1-20 carbon atoms.

5. The method of claim 4 wherein each R group of the solid phase is a butyl group.

6. The method of claim 1 wherein the solid support portion is selected from particles, microparticles and beads.

7. The method of claim 1 wherein the solid support portion comprises an insoluble synthetic polymer.

8. The method of claim 7 wherein the polymer is selected from polystyrene and polyacrylic polymers.

9. The method of claim 1 wherein the solid support portion of the solid phase comprises a glass matrix.

10. The method of claim 1 wherein the solid support portion of the solid phase comprises a silica matrix.

11. The method of claim 1 wherein the cleavable linker portion of the solid phase further comprises one or more connecting portions.

12. The method of claim 1 wherein the solid phase further comprising a magnetically responsive portion.

13. The method of claim 1 wherein the cleavable linker portion of the solid phase is cleaved hydrolytically.

14. The method of claim 13 wherein the hydrolytic cleavage is performed with a solution that contains a base selected from hydroxide salts and alkoxide salts.

15. The method of claim 14 wherein the base is selected from LiOH, NaOH, KOH, NH₄OH, NaOCH₃, KOCH₃, and KOt—Bu.

16. The method of claim 14 wherein the hydrolytic cleavage is performed with a solution that also contains hydrogen peroxide.

17. The method of claim 13 wherein the hydrolytic cleavage is performed with a solution that contains a mineral acid.

18. The method of claim 13 wherein the hydrolytically cleavable linker portion of the solid phase is an ester or thioester group.

19. The method of claim 1 wherein the cleavable linker portion of the solid phase is cleaved reductively.

20. The method of claim 19 wherein the cleavable linker comprises a disulfide or peroxide group.

21. The method of claim 19 wherein the reductive cleavage is performed with a reducing agent selected from thiols, amines and phosphines.

22. The method of claim 21 wherein the reducing agent is selected from ethanethiol, 2-mercaptoethanol, dithiothreitol, a trialkylamine and triphenylphosphine.

23. The method of claim 1 wherein the cleavable linker portion of the solid phase comprises a triggerable dioxetane ring which is cleaved by a triggering agent.

24. The method of claim 23 wherein the triggerable dioxetane has the formula 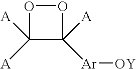
wherein the groups A represent stabilizing substituents. selected from alkyl, cycloalkyl, polycycloalkyl, polycycloalkenyl, aryl, aryloxy and alkoxy groups, Ar represents an aryl ring group which can contain additional substituents selected from halogens, alkoxy and amine groups, Y is a group or atom which is removable by a trigering agent selected from chemical agents and enzymes to cause fragmentation of the dioxetane ring.

25. The method of claim 24 wherein the OY group is selected from OH, OSiR³₃, wherein R³ is selected from alkyl and aryl groups, carboxyl groups, phosphate salts, sulfate salts, and glycoside groups.

26. The method of claim 24 wherein Ar in the triggerable dioxetane is a substituted or unsubstituted phenyl or naphthyl group.

27. The method of claim 23 wherein the triggering agent is selected from bases, fluoride ion, an esterase, a phosphatase, a sulfatase, and a glycosidase.

28. The method of claim 1 wherein the cleavable linker portion of the solid phase comprises an electron rich alkene which is cleaved by conversion to a thermally unstable dioxetane.

29. The method of claim 28 wherein the alkene is converted to the unstable dioxetane by reaction with singlet oxygen.

30. The method of claim 1 wherein the cleavable linker portion of the solid phase is cleaved enzymatically.

31. The method of claim 30 wherein the cleavable linker portion of the solid phase comprises an acridan ketene dithioacetal which is cleaved by reaction with a peroxidase and a peroxide.

32. The method of claim 30 wherein the cleavable linker portion of the solid phase comprises an ester which is cleaved by a hydrolase enzyme or an esterase enzyme.

33. The method of claim 30 wherein the cleavable linker portion of the solid phase comprises an amide which is cleaved by a protease enzyme.

34. The method of claim 30 wherein the cleavable linker portion of the solid phase comprises a peptide which is cleaved by a peptidase enzyme.

35. The method of claim 30 wherein the cleavable linker portion of the solid phase comprises a glycoside which is cleaved by a glycosidase enzyme.

36. The method of claim 13 wherein the cleavable linker portion of the solid phase comprises a thioester having the formula: 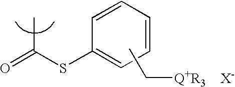
wherein Q is P or N and R is alkyl of 1-20 carbons.

37. The method of claim 36 wherein the cleavable linker portion of the solid phase comprises a thioester having the formula:

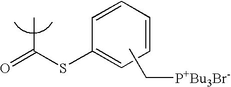

38. The method of claim 1 wherein the cleavable linker portion of the solid phase is an alkylene group of at least one carbon atom bonded to a trialkylphosphonium or triarylphosphonium nucleic acid binding portion and is cleavable by means of a Wittig reaction with a ketone or aldehyde.

39. The method of claim 38 wherein the Wittig reaction forms an ylide by deprotonation with an alkoxide salt or hydride salt base in an aprotic organic solvent and the ylide reacts with a carbonyl compound selected from aliphatic and aromatic aldehydes and aliphatic and aromatic ketones.

40. The method of claim 39 wherein the solvents is selected from THF, diethyl ether, p-dioxane, DMF and DMSO and the carbonyl compound for reaction with the ylide is acetone.

41. The method of claim 38 wherein the cleavable linker portion of the solid phase has the formula

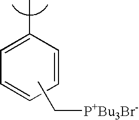

42. The method of claim 1 wherein the cleaving reaction and elution steps are performed as sequential steps using separate and distinct solutions to accomplish each step.

43. The method of claim 1 wherein the cleaving and elution steps can be performed together in the same step.

44. The method of claim 1 further comprising, after step (b), washing the solid phase having captured nucleic acid bound thereto with a wash solution to remove other components of the sample from the solid phase.

45. The method of claim 1 wherein the step of separating the sample from the solid phase is accomplished by magnetic separation.

46. The method of claim 1 wherein the step of separating the sample from the solid phase is accomplished by a process selected from filtration, gravitational settling, decantation, centrifugation, vacuum aspiration, and overpressure of air.

47. The method of claim 2 wherein the nucleic acid binding portion of the solid phase is a ternary sulfonium group of the formula SR₂⁺X⁻ where R is selected from C₁-C₂₀ alkyl, aralkyl and aryl groups, and wherein X is an anion.

48. The method of claim 1 further comprising:
releasing the nucleic acid from the solid phase in step (e) into a solution; and
f) using the solution containing the released nucleic acid directly, in a downstream process.

49. The method of claim 2 further comprising:
releasing the nucleic acid from the solid phase in step (e) into a solution; and
f) using the solution containing the released nucleic acid directly in a downstream process.

50. The method of claim 49 wherein the solution containing the released nucleic acid is used directly in a nucleic acid amplification reaction whereby the amount of the nucleic acid or a segment thereof is amplified using a polymerase or ligase-mediated reaction.
